(19) United States
(12) Patent Application Publication
(10) Pub. No.: US 2011/0123768 A1
(43) Pub. Date: May 26, 2011

(54) PLASTIC FILM, METHOD FOR PRODUCING SAME, POLARIZER AND LIQUID CRYSTAL DISPLAY DEVICE

(75) Inventors: Jun TAKEDA, Minami-ashigara-shi (JP); Keiji Mihayashi, Minami-ashigara-shi (JP)

(73) Assignee: FUJIFILM Corporation, Tokyo (JP)

(21) Appl. No.: 12/954,041

(22) Filed: Nov. 24, 2010

(30) Foreign Application Priority Data
Nov. 25, 2009 (JP) 2009-267390
Sep. 28, 2010 (JP) 2010-217619

(51) Int. Cl. B32B 3/30; C07H 13/02; C08L 1/10; B32B 23/20; B29D 7/01

(52) U.S. Cl. 428/141; 536/63; 106/168.01; 428/536; 264/216

(57) ABSTRACT

A plastic film having an internal haze of at most 0.08 and a total haze of at least 0.41 is, when attached to a polarizer and incorporated in a display device as a retardation film therein, effective for bettering the contrast of the device, and has good film handling characteristics during transport.

### CROSS-REFERENCE TO RELATED APPLICATIONS

The present application claims the benefit of priority from Japanese Patent Application No. 2009-267390, filed on Nov. 25, 2009, and Japanese Patent Application No. 2010-217619, filed on Sep. 28, 2010, the contents of which are herein incorporated by reference in their entirety.

### BACKGROUND OF THE INVENTION

1. Field of the Invention

The present invention relates to a plastic film and its production method, and to a polarizer and a liquid crystal display device using the plastic film. In particular, the invention relates to a plastic film favorable for an optical film such as a retardation film, etc.

2. Description of the Related Art

Heretofore, various plastic films are used, and those with various additives added thereto are known. Plastic films are produced in various film formation methods; and one typical method is a solution casting method where a dope prepared by dissolving a thermoplastic resin in a solvent is cast on a support. For producing a plastic film according to such a solution casting method, there is known a method of using a dope prepared by adding a matting agent thereto for various purposes of improving the film handling characteristics during transport, etc. In the case where the amount of the matting agent added is small, then the produced film may be wrinkled or may give a squeaky feel, therefore often causing a problem of poor film handling characteristics during transport. Accordingly, for example, in producing a cellulose acylate film that is generally known for use as a protective film for polarizer, a matting agent is used as the additive thereto.

Adding a matting agent increases the haze of the film. The film haze generally includes a total haze that means an ordinary haze of film, and an internal haze exclusive of the influence of the roughness of both surfaces of film.

A film having a high total haze may exhibit good adhesiveness when adhered to other film or substrate with an adhesive, and may take a short period of time for saponification when saponified as a pretreatment for adhesion to other film or substrate. Accordingly, from the viewpoint of the application of film and the production cost thereof, films having a high total haze are desired.

On the other hand, films having a high total haze are known to be unfavorable especially for use as optical films.

JP-A 2007-279083 describes a film containing a matting agent, and having an internal haze of at most 0.6% and a surface haze (total haze) of at least 0.05%. This patent publication says that controlling the internal haze and the total haze of a film in that manner enhances the viewing angle characteristics not causing contrast reduction when the film is used in liquid crystal display devices. However, according to Examples and Comparative Examples in the patent publication, the tendency is read that increase in internal haze results in increase in total haze while reduction in internal haze results in reduction in total haze. Specifically, the patent publication does neither substantially suggest nor disclose a film having a low internal haze and a high total haze.

JP-A 2008-262161 discloses a film containing a matting agent and having a total haze of from 0.5 to 0.7. However, this patent publication refers to nothing relating to the level of the internal haze of the film disclosed therein, and does neither disclose nor suggest a method of independently controlling the total haze and the internal haze of the film.

WO2008-54172 discloses a film containing a matting agent and having a total haze of from 0.1 to 0.4, of which the internal haze is controlled to be at most 0.5 times the total haze thereof, concretely disclosing that the internal haze of the film is preferably controlled to fall within a range of from 0.5 to 5 times the total haze thereof. In Comparative Example in the patent publication, the internal haze of the film having a total haze of more than 0.4 is 0.10 or more; and according to the method described in the patent publication, the tendency is read that reduction in the internal haze results in reduction in the total haze. Further, the patent publication discloses nothing relating to the advantage of the film having a total haze of more than 0.41.

The patent publication says that when a retardation film having a lowered internal haze is incorporated in a liquid crystal display device, it can improve the front contrast of device.

As in the above, heretofore, plastic films of which the total haze and the internal haze are both low are disclosed; however, nothing has been investigated relating to a film having a large total haze and having a small internal haze, and in fact, nothing is known relating to a method for producing a film having such specific characteristics.

The present inventors have investigated the film and its production method described in JP-A 2007-279083, and have known that, of the films described in Examples of the patent publication, when a film having an internal haze of at most 0.08 is incorporated into a display device as a retardation film therein, then the film could be effective for enhancing the contrast of the device but the film handling characteristics during transport is still unsatisfactory.

The present inventors have investigated the production method described in JP-A 2008-262161, and have known that, in the case where the plastic film having a total haze of from 0.5 to 0.7 in Example in the patent publication is produced according to the production method described therein, then the internal haze of the film is not lower than 0.12, and when the thus-produced film is attached to a polarizer and incorporated in a display device as a retardation film therein, then the contrast of the device lowers. Accordingly, it is known that the properties and the production method of a plastic film having a high total haze and a low internal haze are not disclosed in JP-A 2008-262161, and further investigation about the plastic film of the type is desired.

The present inventors produced the film described in WO2008-54172, and have known that when the internal haze of the film is lowered then the total haze thereof is also lowered, and that when the film described in the patent publication is attached to a polarizer and incorporated in a display device as a retardation film therein, then the film could be effective for improving the contrast of the device but the film handling characteristics during transport in its production is not good.

### SUMMARY OF THE INVENTION

The present invention is to solve the above-mentioned problems. Specifically, an object of the invention is to provide a plastic film which, when attached to a polarizer and incorporated in a display device, the device could have a good contrast, and the film handling characteristics during transport is good.

Given the situation as above, the present inventors have assiduously studied and, as a result, have found that, when the amount of the matting agent to be added is controlled to fall within a specific range and when it is stretched under a specific condition, then a plastic film, which is effective in enhancing the contrast of a display device when attached to a polarizer and incorporated in a display device as a retardation film therein and which secures good film handling characteristics during transport in its production, can be produced, and have completed the present invention.

Concretely, the inventors have solved the above-mentioned problems, providing the following means:

[1] A plastic film having an internal haze of at most 0.08 and a total haze of at least 0.41 wherein the internal haze is a value measured by using an oil that has a refractive index falling within a range of the refractive index of the thermoplastic resin whose content is the largest in the film, ±0.02, and by coating both surfaces of the film with the oil.
[2] The plastic film of [1], wherein the total haze is at least 0.51.
[3] The plastic film of [1] or [2], having a surface roughness of at least 0.7.
[4] The plastic film of any one of [1] to [3], comprising an ester polymer.
[5] The plastic film of [4], wherein the mean carbon number of the diol residue constituting the ester polymer is from 2.3 to 7.0.
[6] The plastic film of any one of [1] to [5], comprising inorganic fine particles in the outermost layer thereof.
[7] The plastic film of any one of [1] to [6], comprising a cellulose acylate resin or a cyclic olefin resin.
[8] The plastic film of any one of [1] to [6], comprising a cellulose acylate resin.
[9] The plastic film of [7] or [8], wherein the mean value of the degree of total acyl substitution of the cellulose acylate resin is from 2.1 to 2.6.
[10] The plastic film of any one of [1] to [9], comprising a core layer and at least one surface layer laminated on each of both surfaces of the core layer.
[11] The plastic film of [10], produced by co-casting and comprising inorganic fine particles in the surface layer.
[12] The plastic film of [10] or [11], wherein the cellulose acylate resins contained in the core layer differs from the cellulose acylate resin contained in the surface layer in the degree of total acyl substitution.
[13] The plastic film of [12], wherein the cellulose acylate resin constituting the care layer has a degree of total acyl substitution of from 2.1 to 2.6.
[14] The plastic film of any one of [10] to [13], wherein the core layer does not contain inorganic fine particles at all.
[15] A method for producing a plastic film, comprising casting a dope that contains a thermoplastic resin and inorganic fine particles in an amount of at least 0.1% by mass relative to the thermoplastic resin, to thereby form a film to be at least one outermost layer of a plastic film, and stretching the plastic film that contains the film to be the outermost layer thereof, in the direction perpendicular to the film transporting direction (machine direction, film conveying direction) by from 20 to 60% at a stretching temperature A±10° C. wherein A means the temperature at which tan δ of the dynamic viscoelasticity of the thermoplastic resin having a residual solvent content of 0% shows a peak.
[16] The method for producing a plastic film of [15], wherein the thermoplastic resin is a cellulose acylate resin and, in the stretching step, the film is stretched in the direction perpendicular to the film transporting direction by from 20 to 40%.
[17] The method for producing a plastic film of [15] or [16], wherein the dope comprising a thermoplastic resin and inorganic fine particles in an amount of at least 0.1% by mass relative to the thermoplastic resin, and at least one core layer dope comprising a thermoplastic resin are cast successively or co-cast simultaneously in such a manner that the dope comprising a thermoplastic resin and inorganic fine particles in an amount of at least 0.1% by mass relative to the thermoplastic resin can be laminated on both surfaces of the core layer dope.
[18] The method for producing a plastic film of [15] or [16], wherein the dope comprising a thermoplastic resin and inorganic fine particles in an amount of at least 0.1% by mass relative to the thermoplastic resin, and at least one core layer dope comprising a thermoplastic resin are co-cast simultaneously in such a manner that the dope comprising a thermoplastic resin and inorganic fine particles in an amount of at least 0.1% by mass relative to the thermoplastic resin can be laminated on both surfaces of the core layer dope.
[19] The method for producing a plastic film of [17] or [18], wherein the core layer dope does not contain inorganic fine particles at all.
[20] A plastic film produced according to the plastic film production method of any one of [15] to [19].
[21] A polarizer comprising at least one plastic film of any one of [1] to [14] and [20].
[22] A liquid crystal display device comprising at least one plastic film of any one of [1] to [14] and [20].

According to the invention, there is provided a plastic film and its production method, and the plastic film is, when attached to a polarizer and incorporated in a display device as a retardation film therein, effective for bettering the contrast of the device, and has good film handling characteristics during transport in its production.

### BRIEF DESCRIPTION OF DRAWINGS

FIG. 1 is a schematic cross-sectional view of one example of the liquid crystal display device of the invention. In FIG. 1, 11 is polarizing element, 12 is polarizing element, 13 is liquid crystal cell, 14 is cellulose acylate film of Examples and Comparative Examples, and 15 is optically anisotropic film (Fujitac TD80UL).

FIG. 2 is an outline view showing one example of producing a three-layered cellulose acylate laminate film by simultaneous co-casting through a co-casting die. In FIG. 2, 1 is dope for outermost layer (surface layer), 2 is dope for core layer, 3 is co-casting Giesser, and 4 is casting support.

### BEST MODE FOR CARRYING OUT THE INVENTION

Description will now be made in detail of the invention. Although the following description of its structural features may often be made on the basis of typical embodiments of the invention, it is to be understood that the invention is not limited to any such embodiment. It is also to be noted that every numerical range as herein expressed by employing the words “from” and “to”, or simply the word “to”, or the symbol “˜” is supposed to include the lower and upper limits thereof as defined by such words or symbol, unless otherwise noted. In the application, “mass %” means equal to “weight %”, and “% by mass” means equal to “% by weight”.

### [Plastic Film]

The plastic film of the invention, which may be referred to as the film of the invention, has an internal haze of at most 0.08 and a total haze of at least 0.41. The internal haze is a value measured by using an oil that has a refractive index falling within a range of the refractive index of the thermoplastic resin whose content is the largest in the film, ±0.02, and by coating both surfaces of the film with the oil. The film of the invention is described below.

### (Haze)

The film of the invention has an internal haze of at most 0.08. In this description, the internal haze is a value measured by using an oil that has a refractive index falling within a range of the refractive index of the thermoplastic resin whose content is the largest in the film, ±0.02, and by coating both surfaces of the film with the oil.

The film having an internal haze of at most 0.08 is preferred because, when attached to a polarizer and incorporated in a display device, it enhances the contrast of the device. For preventing contrast reduction, the internal haze is more preferably at most 0.07, even more preferably at most 0.06, still more preferably at most 0.05.

The film of the invention has a total haze of at least 0.41, preferably at least 0.51. On the other hand, the uppermost limit of the total haze is preferably at most 1.3, more preferably at most 1.0. When having a total haze of at least 0.41, the film secures smooth sliding on a handling roll in handling for feeding and winding of the roll film, and is therefore hardly scratched and can secure good film handling characteristics during transport. In addition, while stored in the form of a long wound-up roll, the film may be free from a trouble of surface blocking. Further, the adhesiveness of the film to other substrate and film is enhanced, and the time to be taken for saponification of the film may be shortened.

Heretofore, few films are known of which the internal haze and the total haze are independently controlled, and in particular, of films produced according to existing production methods, when the total haze is increased, then the internal haze thereof is also increased, while on the other hand, when the total haze is lowered, then the internal haze thereof is also lowered.

As opposed to this, the invention has realized a plastic film of which the total haze is at least 0.41 and the internal haze is at most 0.08, and has confirmed that when the film is attached to a polarizer and incorporated in a display device, then the contrast of the device is enhanced. In addition, the invention has confirmed that the film having an internal haze of at most 0.08 but having a total haze of at least 0.41 secures good film handling characteristics during transport. Specifically, the film of the invention has a low internal haze and has a high total haze, and is quite different from existing films in that the internal haze and the total haze thereof are controlled in a hitherto-unknown manner, and the film of the invention satisfies both the two requirements of good contrast in its application to polarizer and good film handling characteristics during transport in its production.

### (Surface Roughness)

Preferably, the surface roughness of the film of the invention is at least 0.5 μm from the viewpoint of further bettering the film handling characteristics during transport. More preferably, it is at least 0.65 μm, even more preferably at least 0.7 μm. The surface roughness may be determined according to the following method.

Using Anritsu's film thickness gauge, KG601G, a 5-meter film is analyzed at a pitch of 0.1 mm, and the standard deviation of the measured data is computed. The value is represented by σ, and 6σ is the film surface roughness.

### (Retardation (Re, Rth))

The preferred range of film retardation varies depending on the use of the film. Preferably, the retardation of the film of the invention is 30 nm<Re<100 nm and 80 nm<Rth<300 nm, more preferably, 30 nm<Re<80 nm and 80 nm<Rth<200 nm, even more preferably, 30 nm<Re<70 nm and 80 nm<Rth<150 nm.

In this specification, Re(λ) and Rth(λ) are retardation in plane (nm) and retardation along the thickness direction (nm), respectively, at a wavelength of λ. Re(λ) is measured by applying light having a wavelength of λ nm to a film in the normal direction of the film, using KOBRA 21ADI-1 or WR (by Oji Scientific Instruments).

When a film to be analyze by a monoaxial or biaxial index ellipsoid, Rth(λ) of the film is calculated as follows.

Rth(λ) is calculated by KOBRA 21ADH or WR based on six Re(λ) values which are measured for incoming light of a wavelength λ nm in six directions which are decided by a 10° step rotation from 0° to 50° with respect to the normal direction of a sample film using an in-plane slow axis, which is decided by KOBRA 21ADH, as an inclination axis (a rotation axis; defined in an arbitrary in-plane direction if the film has no slow axis in plane); a value of hypothetical mean refractive index; and a value entered as a thickness value of the film.

In the above, when the film to be analyzed has a direction in which the retardation value is zero at a certain inclination angle, around the in-plane slow axis from the normal direction as the rotation axis, then the retardation value at the inclination angle larger than the inclination angle to give a zero retardation is changed to negative data, and then the Rth(λ) of the film is calculated by KOBRA 21ADH or WR.

Around the slow axis as the inclination angle (rotation angle) of the film (when the film does not have a slow axis, then its rotation axis may be in any in-plane direction of the film), the retardation values are measured in any desired inclined two directions, and based on the data, and the estimated value of the mean refractive index and the inputted film thickness value, Rth may be calculated according to the following formulae (1) and (2):

$$\begin{matrix}Re ( θ ) = [ nx - \frac{ny \times nz}{\sqrt{\begin{matrix}{\{ ny sin ( {sin}^{- 1} ( \frac{sin ( - θ )}{nx} ) ) \}}^{2} + \\ {\{ nz cos ( {sin}^{- 1} ( \frac{sin ( - θ )}{nx} ) ) \}}^{2}\end{matrix}}} ] \times \frac{d}{cos \{ {sin}^{- 1} ( \frac{sin ( - θ )}{nx} ) \}} & Formula ( 1 ) \\ Rth = [ \frac{nx + ny}{2} - nz ] \times d & Formula ( 2 )\end{matrix}$$

wherein Re(θ) represents a retardation value in the direction inclined by an angle θ from the normal direction; nx represents a refractive index in the in-plane slow axis direction; ny represents a refractive index in the in-plane direction perpendicular to nx; nz represents a refractive index in the direction perpendicular to nx and ny; and “d” represents a thickness of the sample.

When the film to be analyzed is not expressed by a monoaxial or biaxial index ellipsoid, or that is, when the film does not have an optical axis, then Rth(λ) of the film may be calculated as follows.

Re (λ) of the film is measured around the slow axis (judged by KOBRA 21ADH or WR) as the in-plane inclination axis (rotation axis), relative to the normal direction of the film from −50 degrees up to +50 degrees at intervals of 10 degrees, in 11 points in all with a light having a wavelength of λ nm applied in the inclined direction; and based on the thus-measured retardation values, the estimated value of the mean refractive index and the inputted film thickness value, Rth(λ) of the film may be calculated by KOBRA 21ADH or WR.

In the above measurement, as the estimated value of the mean refractive index, values in Polymer Handbook (by John Wiley & Sons, Inc.) or those in polymer film catalogues may be used. Materials of which the mean refractive index is unknown may be analyzed with an Abbe's refractiometer to determine their data. For example, the mean refractive index values of some optical films are as follows:

cellulose acylate (1.48), cycloolefin polymer (1.52), polycarbonate (1.59), polymethylmethacrylate (1.49) and polystyrene (1.59).

By inputting the value of these average refraction indices and thickness, KOBRA 21ADH or WR computes nx, ny, nz.

In this specification, the wavelength for measurement is 590 nm unless otherwise specified.

The plastic film of the invention comprises a thermoplastic resin. The plastic film of the invention preferably comprises a cellulose acylate resin or a cyclic olefin resin, more preferably a cellulose acylate resin as the thermoplastic resin.

### (Cellulose Acylate Resin)

The cellulose acylate resins for use in the invention are not specifically defined. The acylate material, cellulose includes cotton linter, wood pulp (hardwood pulp, softwood pulp), etc; and any cellulose acylate from any of such cellulose materials is usable here. As the case may be, two or more of the materials may be mixed for use here. The details of the cellulose materials are described, for example, in Marusawa & Uda's “Lecture of Plastic Materials (17), Cellulose Resins” issued by Nikkan Kogyo Shinbun-sha (1970), or in Hatsumei Kyokai's Disclosure Bulletin No. 2001-1745 (pp. 7-8); and any one described in these is usable here.

The cellulose acylate preferred for use herein is described in detail. The β-1,4-bonding glucose units constituting cellulose have free hydroxyl groups at the 2-, 3- and 6-positions thereof. Cellulose acylate is a polymer prepared by esterifying a part or all of these hydroxyl groups with an acyl group having 2 or more carbon atoms. The degree of acyl substitution means the ratio of esterification of the hydroxyl group in the 2-, 3- and 6-positions of cellulose (100% esterification provides a degree of substitution of 1).

The total degree of acyl substitution, or that is, DS2+DS3+DS6 is preferably from 2.1 to 2.9, more preferably from 2.1 to 2.8. Particularly preferably, the total degree of acyl substitution of the cellulose acylate contained in at least one layer is from 2.1 to 2.6. Also preferably, DS6/(DS2+DS3+DS6) is from 0.08 to 0.66, more preferably from 0.15 to 0.60, even more preferably from 0.20 to 0.45. DS2 means the degree of substitution of the 2-positioned hydroxyl group in the glucose unit with an acyl group (this may be referred to as “degree of 2-acyl substitution below); DS3 means the degree of substitution of the 3-positioned hydroxyl group with an acyl group (this may be referred to as “degree of 3-acyl substitution below); and DS6 means the degree of substitution of the 6-positioned hydroxyl group with an acyl group (this may be referred to as “degree of 6-acyl substitution below). DS6/(DS2

+DS3+DS6) means the proportion of the degree of 6-acyl substitution to the total degree of acyl substitution, and this may be referred to as “proportion of 6-acyl substitution” below.

Only one type of an acyl group, or two or more different types of acyl groups may be in the film of the invention. The film of the invention preferably has an acyl group having from 2 to 4 carbon atoms as the substituent therein. In the case where two or more different types of acyl groups are in the film of the invention, preferably at least one is an acetyl group, and the acyl group having from 2 to 4 carbon atoms is preferably a propionyl group or a butyryl group. The sum total of the degree of substitution of the 2-, 3- and 6-positioned hydroxyl groups with an acetyl group is represented by DSA, and the sum total of the degree of substitution of the 2-, 3- and 6-positioned hydroxyl groups with a propionyl group or a butyryl group is represented by DSB; and the value of DSA+DSB is preferably from 2.3 to 2.6. More preferably, the value of DSA+DSB is from 2.35 to 2.55 and the value of DSB is from 0.10 to 1.70; even more preferably the value of DSA+DSB is from 2.40 to 2.50 and the value of DSB is from 0.5 to 1.2. Controlling the value of DSA and that of DSB to fall within the above range is preferred as providing films of which the fluctuation of Re and Rth is small relative to the environmental humidity.

Specifically, the cellulose acylate resin for use in the invention is preferably a cellulose acylate from the viewpoint of the returnability to nature and of the environmental load.

More preferably, at least 28% of DSB is for the substituent at the 6-positioned hydroxyl group, even more preferably, at least 30% thereof is the substituent at the 6-positioned hydroxyl group, most preferably at least 31% thereof is the substituent at the 6-positioned hydroxyl group, and particularly at least 32% thereof is the substituent at the 6-positioned hydroxyl group. Falling within the range, dopes of higher solubility for films can be prepared, and in particular, good dopes in a chlorine-free solvent can be prepared. Further, dopes having a low viscosity and having good filterability can be prepared.

The acyl group having two or more carbon atoms in the cellulose used in the invention may be an aliphatic group or an aryl group, and are not particularly limited. They may be an alkylcarbonyl ester of cellulose, an alkenylcarbonyl ester of cellulose, an aromatic carbonyl ester of cellulose or an aromatic alkylcarbonyl ester of cellulose. These esters may have a substituent. Preferable examples of the substituents include a propionyl group, a butanoyl group, a heptanoyl group, a hexanoyl group, an octanoyl group, a decanoyl group, a dodecanoyl group, a tridecanoyl group, a tetradecanoyl group, a hexadecanoyl group, an octadecanoyl group, an isobutanoyl group, a tert-butanoyl group, a cyclohexanecarbonyl group, an oleoyl group, a benzoyl group, a naphthylcarbonyl group and a cinnamoyl group. A propionyl group, a butanoyl group, a dodecanoyl group, an octadecanoyl group, a tert-butanoyl group, an oleoyl group, a benzoyl group, a naphthylcarbonyl group and a cinnamoyl group are more preferred, and a propionyl group and a butanoyl group are particularly preferred.

In acylation of cellulose, when an acid anhydride or an acid chloride is used as the acylating agent, the organic solvent as the reaction solvent may be an organic acid, such as acetic acid, or methylene chloride or the like.

When the acylating agent is an acid anhydride, the catalyst is preferably a protic catalyst such as sulfuric acid; and when the acylating agent is an acid chloride (e.g., CH₃CH₂COCl), a basic compound may be used as the catalyst.

A most popular industrial production method for a mixed fatty acid ester of cellulose comprises acylating cellulose with a fatty acid corresponding to an acetyl group and other acyl groups (e.g., acetic acid, propionic acid, valeric acid, etc.), or with a mixed organic acid ingredient containing their acid anhydride.

Cellulose acylate for use in the invention may be produced, for example, according to the method described in JP-A 10-45804.

### (Cyclic Olefin Resin)

The film of the invention may contain a cyclic olefin resin. Not contradictory to the scope and the spirit of the invention, the cyclic olefin resin may be any known cyclic olefin resin; and for example, those described in JP-A 2009-114303 may be used. As the cyclic olefin resin, also usable are commercially-available cyclic olefin resins; and for example, preferred are Arton Series (trade name by JSR, e.g., Model Number, D4531).

### (Film Layer Constitution)

The film of the invention may be composed of one layer or two or more layers.

Preferably, the film of the invention comprises a core layer and at least one surface layer laminated on both surfaces of the core layer. Specifically, the film of the invention preferably has an embodiment of a laminate structure of at least three layers, from the viewpoint of securing the latitude in the process of imparting desired optical properties to the film of the invention that serves as an optical compensatory film. The core layer is a layer having the largest thickness of the film.

In the case where the film of the invention is a laminate film comprising at least 2 layers, preferably, the film is produced by co-casting.

Preferably, the film of the invention contains inorganic fine particles in the surface layer thereof, from the viewpoint of increasing the total haze of the film and increasing the surface roughness of the film.

Also preferably, in the film of the invention, the core layer does not contain inorganic fine particles at all, from the viewpoint of reducing the internal haze of the film.

In the case where the film of the invention is composed of two or more layers, preferably, no adhesive or agglutinant exists between the layers from the viewpoint of simplifying the production process; and the optical film having the layer constitution of the type can be produced according to a lamination casting method to be mentioned below.

The adhesive and the agglutinant to be used in producing a multilayer film in which the constitutive layers are bonded to each other via an adhesive or an agglutinant are described, for example, in JP-A 11-295527.

In the case where the film of the invention contains a cellulose acylate resin as the thermoplastic resin therein and when the film is composed of at least 2 layers, the degree of acyl substitution in the cellulose acylate in each layer may be the same, or different cellulose acylates may be mixed in one layer. Preferably, the mean value of the degree of total acyl substitution of the cellulose acylate resin in the film of the invention is from 2.1 to 2.6, more preferably from 2.2 to 2.5, even more preferably from 2.3 to 2.48.

In the case where the film of the invention is composed of at least 2 layers, more preferably, the mean value of the degree of total acyl substitution of the cellulose acylate resin in the core layer is preferably from 2.1 to 2.6.

More preferably, the mean value of the degree of total acyl substitution of the cellulose acylate resin in the core layer is from 2.2 to 2.5, even more preferably from 2.3 to 2.48.

Preferably, in the film of the invention, the core layer and the surface layer contain cellulose acylate resins differing in the degree of substitution.

The degree of total acyl substitution of the cellulose acylate resin in the surface layer is preferably from 2.6 to 2.9, more preferably from 2.65 to 2.85.

### (Film Thickness)

The thickness of the optical film of the invention (in the case where the optical film consists of two or more layers, the sum of the thickness of the layers) may be suitably defined depending on, for example, the type of the polarizer for which the film is used, but is preferably from 30 to 60 more preferably from 35 to 55 μm. When the film thickness is at most 60 μm, then it is favorable as the production cost may be reduced.

In the case where the film of the invention is composed of two or more layers, the thickness of each layer therein is preferably such that the ratio of the thickness of the surface layer to the total thickness of the film (thickness of the surface layer+thickness of the core layer) is from 0.005 to 0.20, more preferably from 0.005 to 0.15, even more preferably from 0.01 to 0.10.

### <Additive>

Additives such as inorganic fine particles (matting agent), non-phosphate compound, retardation regulator (retardation enhancer, retardation reducer), plasticizer such as phthalate or phosphate compound, UV absorbent, antioxidant and the like may be added to the film of the invention.

### (Inorganic Fine Particles)

Preferably, the film of the invention contains inorganic fine particles at least in the outermost layer thereof.

Preferably, inorganic fine particles (matting agent) are added to the film of the invention. As the inorganic fine particles for use in the invention, there are mentioned silicon dioxide, titanium dioxide, aluminium oxide, zirconium oxide, calcium carbonate, talc, clay, calcined kaolin, calcined calcium carbonate, calcium silicate hydrate, aluminium silicate, magnesium silicate, and calcium phosphate. Silicon-containing inorganic fine particles are preferred for use herein for turbidity reduction, and silicon dioxide is more preferred. The silicon dioxide fine particles preferably have a primary mean particle size of at most 20 nm and an apparent specific gravity of at least 70 g/liter. More preferred are those having a mean particle size of primary particles of from 5 to 30 nm since the total haze of the film can be controlled to fall within the range of the invention. The apparent specific gravity is more preferably from 10 to 100 g/liter or more, even more preferably from 30 to 80 g/liter or more.

In the case where the film of the invention has a laminate structure of at least 2 layers, the inorganic fine particles are contained in at least one outermost layer. When the film of the invention has a laminate structure of at least 3 layers, preferably, the inorganic fine particles are contained in both the two surface layers.

The fine particles generally form secondary particles having a mean particle size of from 0.1 to 3.0 μm; and in the film, the fine particles exist as aggregates of primary particles and form irregularities of from 0.1 to 3.0 μm in size in the film surface. The secondary mean particle size is preferably from 0.2 μm to 1.5 μm. more preferably from 0.4 μm to 1.2 μm, most preferably from 0.6 μm to 1.1 μm. Regarding the size of the primary and secondary particles, the particles in the film are observed with a scanning electronic microscope, and the diameter of the circle circumscribing around the particle is taken as the particle size. 200 particles selected at random are observed and measured, and their data are averaged to give the mean particle size.

As the silicon dioxide fine particles, for example, herein usable are commercial products of Aerosil 8972, R972V, R974, R812, 200, 200V, 300, 8202, OX50, TT600 (all by Nippon Aerosil), etc. Zirconium oxide fine particles are available as commercial products of Aerosil R976 and R811 (trade names by Nippon Aerosil), and these are usable here.

Of those, Aerosil R972 is especially preferred from the viewpoint of the aggregation behavior thereof in preparing a dispersion of inorganic fine particles.

In the invention, in order to produce a film containing particles having a small secondary mean particle size, some methods may be taken into consideration in preparing the dispersion of fine particles. For example, one method is as follows: A solvent and fine particles are previously stirred and mixed to prepare a dispersion of fine particles, and the dispersion of fine particles is added to a small amount of a cellulose acylate solution separately prepared, and stirred and dissolved therein, and this is further mixed with a main cellulose acylate dope liquid. In this method, silicon dioxide fine particles well disperse and hardly reaggregate, and therefore the method is preferred. Another method is as follows: A small amount of cellulose ester is added to a solvent, and stirred and dissolved, and then fine particles are added thereto and dispersed with a disperser to prepare a fine particles-added liquid, and the fine particles-added liquid is well mixed with a dope liquid in an in-line mixer. The invention is not limited to these methods. In mixing and dispersing silicon dioxide fine particles in a solvent in the invention, the concentration of silicon dioxide is preferably from 5 to 30% by mass, more preferably from 10 to 25% by mass, most preferably from 15 to 20% by mass. The dispersion concentration is preferably higher, since the liquid turbidity relative to the added amount is lower, and therefore the haze could fall within a specific range and the aggregates are hardly formed. The amount of the matting agent to be in the final cellulose acylate dope liquid is preferably from 0.01 to 1.0 g/m², more preferably from 0.03 to 0.3 g/m², even more preferably from 0.08 to 0.16 g/m².

Lower alcohols are usable as the solvent here, including, for example, methyl alcohol, ethyl alcohol, propyl alcohol, isopropyl alcohol, butyl alcohol, etc. Not specifically defined, other solvents than lower alcohols are also usable, for which, for example, the solvent for use in cellulose ester film formation is preferred.

### (Non-Phosphate Compound)

Preferably, the film of the invention contains a phosphate compound or a non-phosphate polyester compound for preventing the phenomenon of additive bleeding from the film in wet heat condition. The additives for use in the film of the invention are described in detail hereinunder.

The film of the invention preferably contains a non-phosphate compound in the low-substitution layer. The non-phosphate compound in the layer exhibits an effect of preventing whitening.

In this description, the “non-phosphate compound” means “a compound having an ester bond in which the acid contributing to the ester bond is one except phosphoric acid”. In other words, the “non-phosphate compound” means an ester compound not containing phosphoric acid.

The non-phosphate compound may be a low-molecular compound or a polymer (high-molecular compound). The non-phosphate compound in the form of a polymer may be referred to as a non-phosphate polymer below.

As the non-phosphate compound, widely usable are high-molecular additives and low-molecular additives known as additives to cellulose acylate films. Preferably, the amount of the additive is from 1 to 35% by mass of the cellulose resin, more preferably from 4 to 30% by mass, even more preferably from 10 to 25% by mass.

The polymer additive for use in the film of the invention as a non-phosphate compound therein has a recurring unit in the compound, and preferably has a viscosity-average molecular weight of from 600 to 10000. The polymer additive has the function of promoting the solvent evaporation speed in solution casting to form the film, and the function of reducing the residual solvent amount in the film. Further, the compound exhibits other various useful effects of film quality improvement of, for example, enhancing the mechanical property of film, imparting flexibility, imparting moisture absorption resistance, reducing the moisture permeability, etc.

The viscosity-average molecular weight of the high-molecular additive of non-phosphate compound for use in the invention is more preferably from 600 to 8000, even more preferably from 600 to 5000, still more preferably from 600 to 5000.

The high-molecular additive of non-phosphate compound for use in the invention is described in detail below with reference to specific examples thereof given below; however, needless-to-say, the high-molecular additive of non-phosphate compound for use in the invention is not limited to these.

The polymer additive of non-phosphate compound includes polyester polymer (aliphatic polyester polymer, aromatic polyester polymer, etc.), and copolymer of polyester ingredient and other ingredient, etc. Preferred are aliphatic polyester polymer, aromatic polyester polymer; copolymer of polyester polymer (aliphatic polyester polymer, aromatic polyester polymer, etc.) and acrylic polymer; and copolymer of polyester polymer (aliphatic polyester polymer, aromatic polyester polymer, etc.) and styrenic polymer. More preferred are polyester compounds containing an aromatic ring as at least one copolymerization ingredient.

The aliphatic polyester-type polymers for use in the invention is one produced by reaction of a mixture of an aliphatic dicarboxylic acid having from 2 to 20 carbon atoms, and a diol selected from the group consisting of aliphatic dials having from 2 to 12 carbon atoms and alkyl ether diols having from 4 to 20 carbon atoms, and both ends of the reaction product may be as such, or may be blocked by further reaction with a monocarboxylic acid or a monoalcohol. The terminal blocking may be effected for the reason that the absence of a free carboxylic acid in the plasticizer is effective for the storability of the plasticizer. The dicarboxylic acid for the polyester plasticizer for use in the invention is preferably an aliphatic dicarboxylic acid having from 4 to 20 carbon atoms or an aromatic dicarboxylic acid having from 8 to 20 carbon atoms.

The aliphatic dicarboxylic acids having from 2 to 20 carbon atoms preferably for use in the film of the invention include, for example, oxalic acid, malonic acid, succinic acid, maleic acid, fumaric acid, glutaric acid, adipic acid, pimelic acid, suberic acid, azelaic acid, sebacic acid, dodecanedicarboxylic acid and 1,4-cyclohexanedicarboxylic acid.

More preferred aliphatic dicarboxylic acids in these are malonic acid, succinic acid, maleic acid, fumaric acid, glutaric acid, adipic acid, azelaic acid, 1,4-cyclohexanedicarboxylic acid. Particularly preferred dicarboxylic acids are succinic acid, glutaric acid and adipic acid.

The diol used for the high-molecular additive are selected, for example, from aliphatic diols having from 2 to 20 carbon atoms, alkyl ether dials having from 4 to 20 carbon atoms.

Examples of the aliphatic diol having from 2 to 20 carbon atoms include an alkyldiol and an aliphatic dial. For example, an ethandiol, 1,2-propandiol, 1,3-propandiol, 1,2-butandiol, 1,3-butandiol 2-methyl-1,3-propandiol, 1,4-butandiol 1,5-pentandiol, 2,2-dimethyl-1,3-propandiol (neopentyl glycol), 2,2-diethyl-1,3-propandial (3,3-dimethylolpentane), 2-n-buthyl-2-ethyl-1,3-propandiol (3,3-dimethylolheptane), 3-methyl-1,5-pentandiol, 1,6-hexandiol, 2,2,4-trimethyl-1,3-pentandiol, 2-ethyl-1,3-hexandiol, 2-methyl-1,8-octandiol, 1,9-nonandiol, 1,10-decandiol, 1,12-octadecandiol, etc. One or more of these glycols may be used either singly or as combined mixture.

Specific examples of preferred aliphatic diols include an ethandiol, 1,2-propandiol, 1,3-propandiol, 1,2-butandiol, 1,3-butandiol, 2-methyl-1,3-propandiol, 1,4-butandiol, 1,5-pentandiol, 3-methyl-1,5-pentandiol, 1,6-hexandiol, 1,4-cyclohexandiol, 1,4-cyclohexandimethanol. Particularly preferred examples include ethandiol, 1,2-propandiol, 1,3-propandiol, 1,2-butandiol, 1,3-butandiol, 1,4-butandiol, 1,5-pentandiol, 1,6-hexandiol, 1,4-cyclohexandiol, 1,4-cyclohexanedimethanol.

Specific examples of preferred alkyl ether diols having from 4 to 20 carbon atoms are polytetramethylene ether glycol, polyethylene ether glycol and polypropylene ether glycol, and combinations of these. The average degree of polymerization is not limited in particular, and it is preferably from 2 to 20, more preferably 2 to 10, further preferably from 2 to 5, especially preferably from 2 to 4. As these examples, Carbowax resin, Pluronics resin and Niax resin are commercially available as typically useful polyether glycols.

In the invention, an aliphatic polyester polymer in which the mean carbon number of the diol residue is at least 2.3 is preferred as the additive to the film, as enhancing the durability of the film. The mean carbon number of the diol residue is more preferably at least 2.4, even more preferably at least 2.5. The upper limit of the mean carbon number is preferably at most 7.0. The mean carbon number of the diol residue as referred to herein means the mean value of the carbon number of the diol residue that constitutes the polymer. In the case where two or more dial ingredients are used in producing the aliphatic polyester polymer, the carbon number of each diol ingredient is multiplied by the molar fraction and all the data are summed up to give the mean carbon number. One typical example of the case where two or more dial ingredients are combined is a combination of ethylene glycol and glycol having at least 3 carbon atoms.

Preferably, the viscosity-average molecular weight of the aliphatic polyester polymer for use in the invention is from 600 to 2000, more preferably from 600 to 1400, even more preferably from 600 to 900.

In the invention, especially preferred is a high-molecular additive of which the terminal is blocked with an alkyl group or an aromatic group. The terminal protection with a hydrophobic functional group is effective against aging at high temperature and high humidity, by which the hydrolysis of the ester group is retarded.

Preferably, the polyester plasticizer in the invention is protected with a monoalcohol residue or a monocarboxylic acid residue in order that both ends of the polyester plasticizer are not a carboxylic acid or a hydroxyl group.

In this case, the monoalcohol residue is preferably a substituted or unsubstituted monoalcohol residue having from 1 to 30 carbon atoms, including, for example, aliphatic alcohols such as methanol, ethanol, propanol, isopropanol, butanol, isobutanol, pentanol, isopentanol, hexanol, isohexanol, cyclohexyl alcohol, octanol, isooctanol, 2-ethylhexyl alcohol, nonyl alcohol, isononyl alcohol, tert-nonyl alcohol, decanol, dodecanol, dodecahexanol, dodecaoctanol, allyl alcohol, oleyl alcohol; and substituted alcohols such as benzyl alcohol, 3-phenylpropanol.

Alcohol residues for terminal blocking that are preferred for use in the invention are methanol, ethanol, propanol, isopropanol, butanol, isobutanol, isopentanol, hexanol, isohexanol, cyclohexyl alcohol, isooctanol, 2-ethylhexyl alcohol, isononyl alcohol, oleyl alcohol, benzyl alcohol, more preferably methanol, ethanol, propanol, isobutanol, cyclohexyl alcohol, 2-ethylhexyl alcohol, isononyl alcohol, benzyl alcohol.

In blocking with a monocarboxylic acid residue, the monocarboxylic acid for use as the monocarboxylic acid residue is preferably a substituted or unsubstituted monocarboxylic acid having from 1 to 30 carbon atoms. It may be an aliphatic monocarboxylic acid or an aromatic monocarboxylic acid. Preferred aliphatic monocarboxylic acids are described. They include acetic acid, propionic acid, butanoic acid, caprylic acid, caproic acid, decanoic acid, dodecanoic acid, stearic acid, oleic acid. Preferred aromatic monocarboxylic acids are, for example, benzoic acid, p-tert-butylbenzoic acid, orthotoluic acid, metatoluic acid, paratoluic acid, dimethylbenzoic acid, ethylbenzoic acid, normal-propylbenzoic acid, aminobenzoic acid, acetoxybenzoic acid. One or more of these may be used either singly or as combined.

The high-molecular additive for use in the invention may be easily produced according to any of a thermal melt condensation method of polyesterification or interesterification of the above-mentioned dicarboxylic acid and diol and/or monocarboxylic acid or monoalcohol for terminal blocking, or according to an interfacial condensation method of an acid chloride of those acids and a glycol in an ordinary manner. The polyester additives are described in detail in Koichi Murai's “Additives, Their Theory and Application” (by Miyuki Publishing, first original edition published on Mar. 1, 1973). The materials described in JP-A 05-155809, 05-155810, 05-197073, 2006-259494, 07-330670, 2006-342227, 2007-003679 are also usable herein.

The aromatic polyester polymers are obtained by copolymerizing the above-mentioned polyester polymers with a monomer having an aromatic ring. The monomer having an aromatic ring is at least one monomer selected from aromatic dicarboxylic acids having from 8 to 20 carbon atoms, and aromatic diols having from 6 to 20 carbon atoms.

The aromatic dicarboxylic acids for use in the film of the invention having from 8 to 20 carbon atoms include phthalic acid, terephthalic acid, isophthalic acid, 1,5-naphthalene dicarboxylic acid, 1,4-naphthalene dicarboxylic acid, 1,8-naphthalene dicarboxylic acid, 2,8-naphthalene dicarboxylic acid and 2,6-naphthalene dicarboxylic acid etc. Preferable aromatic dicarboxylic acids are phthalic acid, terephthalic acid and isophthalic acid.

The aromatic diols having from 6 to 20 carbon atoms, not limited, include Eiisphenol A, 1,2-hydroxybenzene, 1,3-hydroxybenzene, 1,4-hydroxybenzene, 1,4-dimethylolbenzene, and preferably include bisphenol A, 1,4-hydroxybenzene and 1,4-dimethylolbenzene.

In the invention, the aromatic polyester polyester is combined with at least one of aromatic dicarboxylic acids or aromatic diols, and the combination is not specifically defined. Different types of the respective ingredients may be combined with no problem. In the invention, especially preferred are high-molecular-weight additives the terminal of which is blocked with an alkyl group or an aromatic group, as so mentioned in the above; and for the blacking, the above-mentioned method may be employed.

For example, phosphate compounds and non-ester additives known as additives to cellulose acylate film can be widely used in the invention as a retardation reducer other than non-phosphate compounds.

The polymer-type retardation reducer may be selected from phosphate polyester polymers, styrenic polymers, acrylic polymers and their copolymers; and acrylic polymers and styrenic polymers are preferred. Preferably, the retardation reducer contains at least one polymer having a negative intrinsic birefringence such as styrenic polymer and acrylic polymer.

The low-molecular weight retardation reducer except non-phosphate compounds includes the following. These may be solid or oily. In other words, they are not specifically defined in point of the melting point or boiling point thereof. For example, there is mentioned mixing UV-absorbent materials having a melting point of 20° C. or less, or having a melting point of 20° C. or more, as well as mixing antiaging agents similarly. IR absorbent dyes are described in, for example, JP-A 2001-194522. The additive may be added in any stage of preparing the cellulose acylate solution (dope); and the additive may be added at the end of the dope preparation process in the final step for additive addition of the process. The amount of the material is not specifically defined so far as the material could exhibit its function.

The low-molecular retardation reducer of compounds except non-phosphate compounds is not specifically defined. For example, the compounds are described in detail in JP-A 2007-272177, paragraphs [0066] to [0085].

The compounds represented by a general formula (1) in JP-A 2007-272177, paragraphs [0066] to [0085] may be produced according to the following method.

The compounds of formula (1) in the patent publication can be produced by condensation of a sulfonyl chloride derivative and an amine derivative.

The compounds of a general formula (2) in JP-A 2007-272177 can be produced by dehydrating condensation of a carboxylic acid and an amine with a condensing agent (e.g., dicyclohexylcarbodiimide (DCC), etc.), or by substitution reaction between a carboxylic acid chloride derivative and an amine derivative.

The retardation reducer in the invention is preferably an Rth reducer from the viewpoint of realizing a favorable Nz factor. Of the retardation reducers, the Rth reducer includes, for example, acrylic polymers, styrenic polymers, and low-molecular-weight compounds of formulae (3) to (7) of JP-A 2007-272177. Of those, preferred are acrylic polymers and styrenic polymers; and more preferred are acrylic polymers.

The retardation reducing agent is added in an amount of preferably from 0.01 to 30% by mass of the cellulose resin, more preferably from 0.1 to 20% by mass of the cellulose resin, still more preferably from 0.1 to 10% by mass of the cellulose resin.

When the retardation reducing agent is added in an amount of at most 30% by mass, compatibility with the cellulose resin can be improved and whitening can be inhibited. When two or more retardation reducing agents are used, the sum amount of the agents is preferably within the above range.

### (Plasticizer)

Many compounds known for a plasticizer of a cellulose acylate may be preferably used as a plasticizer in the invention. As the plasticizer, usable are phosphates or carboxylates. Examples of the phosphates include triphenyl phosphate (TPP) and tricresyl phosphate (TCP). The carboxylates are typically phthalates and citrates. Examples of the phthalate compounds include dimethyl phthalate (DMP), diethyl phthalate (DEP), dibutyl phthalate (DPP), dioctyl phthalate (DOP), diphenyl phthalate (DPP) and diethylhexyl phthalate (DEHP). Examples of the citrates include triethyl O-acetylcitrate (OACTE) and tributyl O-acetylcitrate (OACTB). Examples of other carboxylates include butyl oleate, methylacetyl ricinoleate, dibutyl sebacate, and various trimellitates. Preferred for use herein are phthalate plasticizers (DMP, DEP, DBP, DOP, DPP, DEEP). More preferred are DEP and DPP.

### (Retardation Enhancer)

The film of the invention may contain a retardation enhancer. Containing a retardation enhancer, the film may express high Re expressibility at a low draw ratio in stretching it. The type of the retardation enhancer for use herein is not specifically defined. There may be mentioned retardation-enhancing, rod-shaped or discotic compounds and non-phosphate compounds. Of such rod-shaped or discotic compounds, those having at least two aromatic rings are preferred for the retardation enhancer for use herein.

Two or more different types of retardation enhancers may be combined for use herein.

Preferably, the retardation enhancer has a maximum absorption in a wavelength region of from 250 to 400 nm, and does not substantially have an absorption in a visible light region.

As the retardation enhancer, for example, the compounds described in JP-A 2004-50516 and 2007-86748 are usable here, to which, however, the invention should not be limited.

As the discotic compound, for example, preferred for use herein are the compounds described in EP 0911656A2, the triazine compounds described in JP-A 2003-344655, the triphenylene compounds described in JP-A 2008-150592, paragraphs [0097] to [0108].

The discotic compounds may be produced according to known methods, for example, according to the method described in JP-A 2003-344655 or the method described in JP-A 2005-134884.

Apart from the above-mentioned discotic compounds, rod-shaped compounds having a linear molecular structure are also preferably used here, and for example, the rod-shaped compounds described in JP-A 2008-150592, paragraphs [0110] to are preferred for use here.

Two or more different types of rod-shaped compounds may be combined for use herein, of which the maximum absorption wavelength (λmax) thereof is longer than 250 nm in the UV absorption spectrum of the solution of the compound.

The rod-shaped compounds may be produced with reference to the methods described in literature. The literature includes Mol. Cryst. Liq. Cryst., Vol. 53, p. 229 (1979); ibid., Vol. 89, p. 93 (1982); ibid., Vol. 145, p. 111 (1987); ibid., Vol. 170, p. 43 (1989); J. Am. Chem. Soc., Vol. 113, p. 1349 (1991); ibid., Vol. 118, p. 5346 (1996); ibid., Vol. 92, p. 1582 (1970); J. Org. Chem., Vol. 40, p. 420 (1975); Tetrahedron, Vol. 48, No. 16, p. 3437 (1992).

### (Polarizer)

The film of the invention is applicable to a polarizer, which comprises at least one film of the invention.

The polarizer of the invention preferably comprises a polarizing element and the film of the invention on one side of the polarizing element. Like the optical compensatory film of the invention, the embodiment of the polarizer includes not only an embodiment of a polarizer in the form of a film cut in a size capable of being directly incorporated in a liquid crystal display device but also an embodiment of a polarizer in the form of a long-size, rolled film (for example, an embodiment having a rolled length of 2500 m or more, or 3900 m or more). In order to be applicable to a large-panel liquid crystal display device, the width of the polarizer is preferably at least 1470 mm, as so mentioned in the above.

Regarding the constitution of the polarizer, there is no specific limitation thereon but a known constitution is employable. For example, the constitution of FIG. 6 in JP-A 2008-262161 is employable here.

### (Liquid Crystal Display Device)

The film of the invention is applicable to the liquid crystal display device comprising the above-mentioned polarizer.

The liquid crystal display device of the invention is a liquid crystal display device comprising a liquid crystal cell and a pair of polarizers arranged on both sides of the liquid crystal cell, in which at lest one polarizer is the polarizer of the invention. Preferably, the device is an IPS-mode, CCS-mode or VA-mode liquid crystal display device.

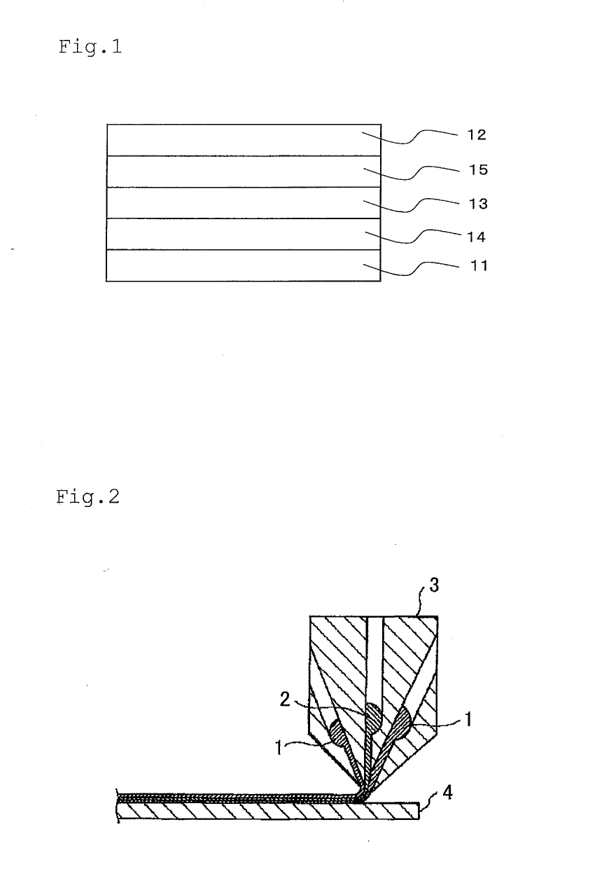

Regarding the constitution of the liquid crystal display device, there is no specific limitation thereon but a known constitution is employable. For example, the constitution of FIG. 1 is employable, or the constitution of FIG. 2 in JP-A 2008-262161 is also preferred.

### [Production Method for Optical Film]

The production method for the optical film of the invention (this may be referred to as the production method of the invention below) comprises a step of casting a dope that contains a thermoplastic resin and inorganic fine particles in an amount of at least 0.1% by mass relative to the thermoplastic resin, to thereby form a film to be at least one outermost layer of a plastic film, and a step of stretching the plastic film that contains the film to be the outermost layer thereof, in the direction perpendicular to the film transporting direction by from 20 to 60% at a stretching temperature A±10° C. wherein A means the temperature at which tan δ of the dynamic viscoelasticity of the thermoplastic resin having a residual solvent content of 0% shows a peak.

According to the production method of the invention, an unknown plastic film having a low internal haze and a high total haze, which are adjusted by an unknown embodiment, can be produced.

The film of the invention is produced according to a solution casting method (solvent casting method). For production examples of cellulose acylate films according to a solvent casting method, for example, referred to are U.S. Pat. Nos. 2,336,310, 2,367,603, 2,492,078, 2,492,977, 2,492,978, 2,607,704, 2,739,069 and 2,739,070; BP 640731 and 736892; JP-B 45-4554 and 49-5614; JP-A 60-176834, 60-203430 and 62-115035. The film of the invention is stretched. The method and the condition for stretching treatment other than those described in this specification are described, for example, in JP-A 62-115035, 4-152125, 4-284211, 4-298310 and 11-48271.

### <Preparation of Dope>

In the solvent casting method, a solution (dope) prepared by dissolving a thermoplastic resin in an organic solvent is used for film production.

The organic solvent preferably contains an organic solvent selected from an ether having from 3 to 12 carbon atoms, a ketone having from 3 to 12 carbon atoms, an ester having from 3 to 12 carbon atoms, and a halogenohydrocarbon having from 1 to 6 carbon atoms. The ether, ketone and ester may have a cyclic structure. A compound having at least two functional groups of ether, ketone and ester (i.e., —O—, —CO— and —COO—) is also usable as the organic solvent. The organic solvent may have any other functional group such as an alcoholic hydroxyl group. When the organic solvent has two or more different types of functional groups, the number of the carbon atoms constituting the group may fall within the range defined for the compound having the functional group.

Examples of the ether having from 3 to 12 carbon atoms include diisopropyl ether, dimethoxymethane, dimethoxyethane, 1,4-dioxane, 1,3-dioxolan, tetrahydrofuran, anisole and phenetole.

Examples of the ketone having from 3 to 12 carbon atoms include acetone, methyl ethyl ketone, diethyl ketone, diisobutyl ketone, cyclohexane and methylcyclohexanone.

Examples of the ester having from 3 to 12 carbon atoms include ethyl formate, propyl formate, pentyl formate, methyl acetate, ethyl acetate and pentyl acetate.

Examples of the organic solvent having two or more different types of functional groups include 2-ethoxyethyl acetate, 2-methoxyethanol and 2-butoxyethanol.

The number of the carbon atoms constituting the halogenohydrocarbon is preferably 1 or 2, most preferably one. Preferably, the halogen of the halogenohydrocarbon is chlorine. The proportion of substitution of the hydrogen atom in the halogenohydrocarbon with halogen is preferably from 25 to 75 mol %, more preferably from 30 to 70 mol %, even more preferably from 35 to 65 mol %, most preferably from 40 to 60 mol %. Methylene chloride is a typical halogenohydrocarbon.

Two or more different types of organic solvents may be mixed for use herein.

The thermoplastic resin solution may be prepared according to an ordinary method. The ordinary method is meant to include treatment at a temperature of 0° C. or higher (room temperature or high temperature). The solution may be prepared according to a dope preparation method and using a dope preparation apparatus in an ordinary solvent casting method. In the ordinary method, a halogenohydrocarbon (especially methylene chloride) is preferred for the organic solvent.

The amount of the thermoplastic resin is so controlled that the prepared solution could contain it in an amount of from 10 to 40% by mass. More preferably, the amount of the thermoplastic resin is from 10 to 30% by mass. The preferred range of the thermoplastic resin is the same as the preferred range thereof in the film of the invention, and the resin is preferably a cellulose acylate resin or a cyclic olefin resin, more preferably a cellulose acylate resin, particularly preferably a cellulose acetate resin. The organic solvent (main solvent) may contain any additive of the above-mentioned additives that may be preferably in the film of the invention. In the production method of the invention, the dope preferably contains inorganic particles.

The solution may be prepared by stirring a thermoplastic resin and an organic solvent at room temperature (0 to 40° C.). A high-concentration solution may be stirred under pressure and under heat. Concretely, a thermoplastic resin and an organic solvent are put in a pressure container and sealed up, and these are stirred under pressure and under heat at a temperature higher than room temperature but not higher than the boiling point of the organic solvent. The heating temperature is generally 40° C. or higher, preferably from 60 to 200° C., more preferably from 80 to 110° C.

The ingredients may be put in a reactor after they are roughly premixed. They may be sequentially put in a reactor. The reactor must be so designed that the contents therein could be stirred. An inert gas such as nitrogen gas may be introduced into the reactor to increase the pressure therein. The increase in the vapor pressure by heating may be utilized. Alternatively, after the reactor is sealed up, the constitutive ingredients may be put therein under pressure.

In case where the ingredients are heated, preferably, they are heated from the outside of the reactor. For example, a jacket-type heating apparatus may be used. A plate heater with a duct running therein may be arranged around the reactor, and a liquid may be circulated in the duct, whereby the reactor may be heated as a whole.

Preferably, a stirring blade is arranged inside the reactor, and the contents therein are stirred with it. Preferably, the stirring blade has a length that reaches around the wall of the reactor. Preferably, the tip of the stirring blade is provided with a scraper for renewing the liquid film around the wall of the reactor.

Instruments such as a pressure gauge, a thermometer and the like may be arranged in the reactor. In the reactor, the constitutive ingredients are dissolved in a solvent. The prepared dope may be taken out of the reactor after cooled therein, or may be taken out and then cooled with a heat exchanger or the like.

### <Casting Method>

As the solution casting method, there are known an extrusion method of uniformly extruding a prepared dope from a pressure die onto a metal support, a doctor blade method where the dope once cast on a metal support is treated with a blade to control its thickness, and a reverse roll method of controlling a once-cast dope with a reverse roll coater. Preferred is the method through a pressure die. The pressure die includes a coat hanger type one or a T-die type one, and any of these is preferably used here. Apart from the methods mentioned herein, any other various known solution-casting methods using various known cellulose triacetate solutions may be employed here. Taking the difference in boiling point and others between the solvents to be used into consideration and defining various conditions, various solution casting methods may be effected to attain the same effects as those described in the related references.

In the production method of the invention, a dope comprising a thermoplastic resin and inorganic fine particles in an amount of at least 0.1% by mass relative to the thermoplastic resin is cast to form a film to be at least one outermost layer of the plastic film, in order that the total haze of the plastic film could fall within the range of the invention. The film to be the outermost layer is the film of the invention directly as it is, when the film of the invention is a single-layered film. On the other hand, when the film of the invention is composed of 2 or more layers, the above film to be the outermost layer is at least one outermost layer of the film of the invention, and preferably, it is to be the outermost layer on both sides of the film of the invention.

### (Co-Casting)

In producing the film of the invention comprising two or more layers, preferred is a lamination casting method of a co-casting method, a successive-casting method, a coating method or the like. More preferred is a simultaneous co-casting method or a successive-casting method; and even more preferred is a simultaneous co-casting method from the viewpoint of stable production and production cost reduction.

In the case where the film of the invention is produced according to a co-casting method or a successive-casting method, the thermoplastic resin solution (dope) for each layer is first prepared.

In the production method of the invention, preferably, the dope containing a thermoplastic resin and inorganic fine particles in an amount of at least 0.1% by mass relative to the thermoplastic resin, and at least one core layer dope containing a thermoplastic resin are cast successively or co-cast simultaneously in such a manner that the dope containing a thermoplastic resin and inorganic fine particles in an amount of at least 0.1% by mass relative to the thermoplastic resin can be laminated on both surfaces of the core layer dope.

In the production method of the invention, more preferably, the dope containing a thermoplastic resin and inorganic fine particles in an amount of at least 0.1% by mass relative to the thermoplastic resin, and at least one core layer dope containing a thermoplastic resin are co-cast simultaneously in such a manner that the dope containing a thermoplastic resin and inorganic fine particles in an amount of at least 0.1% by mass relative to the thermoplastic resin can be laminated on both surfaces of the core layer dope.

In the case where a cellulose acylate resin is used as the thermoplastic resin in the production method of the invention, preferably, the dope containing a thermoplastic resin and inorganic fine particles in an amount of at least 0.1% by mass relative to the thermoplastic resin and the core layer dope contain cellulose acylate resins differing in the degree of substitution.

In the production method of the invention, preferably, the mean value of the degree of total acyl substitution of the cellulose acylate resin in the core layer dope is preferably from 2.1 to 2.6. The more preferred range of the value is the same as that described hereinabove in the section of the description of the film of the invention.

In the simultaneous co-casting method (multilayer simultaneous casting method), casting dopes are simultaneously extruded out through a casting Giesser through which the individual casting dopes for the intended layers (two or more layers) are simultaneously cast via different slits onto a casting metal support (band or drum), and at a suitable time, the film formed on the metal support is peeled away and dried. FIG. 2 is a cross-sectional view showing a mode of simultaneous extrusion to form three layers by casting the dope 1 for surface layer and the dope 2 for core layer on a casting metal support 4 through a co-casting Giesser 3.

The successive-casting method is as follows: First the dope for outermost layer is extruded out and cast onto a casting metal support through a casting Giesser, then after it is dried or not dried, the casting dope for second layer (core layer) is cast onto it in a mode of extrusion through a casting Giesser, and if desired, three or more layers are successively formed in the same mode of casting and lamination, and at a suitable time, the resulting laminate film is peeled away from the metal support and dried.

On the other hand, the coating method is as follows: A film of a core layer is formed according to a solution casting method, then a coating solution for surface layer is prepared, and using a suitable coater, the coating solution is applied onto the previously formed core film first on one surface thereof and next on the other surface thereof, or simultaneously on both surfaces thereof, and the resulting laminate film is dried.

For example, dopes differing in the concentration of the additives therein such as plasticizer, UV absorbent and inorganic fine particles (matting agent) may be co-cast to produce a plastic film having a laminate structure in which the constitutive layers have a different additive concentration. The concentration of the inorganic fine particles may be higher in the surface layer dope and may be low in the core layer dope. In the production method of the invention, preferably, the surface layer dope contains inorganic fine particles and, more preferably, the core layer dope does not contain inorganic fine particles at all from the viewpoint of reducing the internal haze of the produced film.

The amount of the plasticizer and the UV absorbent may be higher in the core layer dope than in the surface layer dope, and the additives may be only in the core layer dope. The type of the plasticizer and the UV absorbent may differ between the core layer dope and the surface layer dope. In a preferred embodiment, a release agent may be added to only the surface layer dope on the side of the metal support. In order to cool the metal support and to gel the dope according to a cooling drum method, preferably, the amount of the poor solvent, alcohol in the surface layer dope may be larger than in the core layer dope. The surface layer dope and the core layer dope may differ in Tg; and preferably, Tg of the core layer dope is lower than that of the surface layer dope. In casting, the viscosity of the dope containing a thermoplastic resin may differ between the surface layer dope and the core layer dope; and preferably, the viscosity of the surface layer dope is smaller than the viscosity of the core layer dope; however, the viscosity of the core layer dope may be smaller than that of the surface layer dope.

In the production method of the invention, preferably, the thermoplastic resin differs between the surface layer dope and the core layer dope. In the case where a cellulose acylate resin is sued as the thermoplastic resin, preferably, the cellulose acylate resin differs in the degree of total acyl substitution between the surface layer dope and the core layer dope.

As the endlessly running metal support for use in producing the film of the invention, preferably usable is a drum of which the surface is mirror-finished by chromium plating, or a. SUS (stainless) belt (band) of which the surface is mirror-finished by polishing. One or more pressure dies may be arranged above the metal support. Preferably, one or two pressure dies are arranged. In case where two or more pressure dies are arranged, the dope to be cast may be divided into portions suitable for the individual dies; or the dope may be fed to the die at a suitable proportion via a plurality of precision metering gear pumps. The temperature of the cellulose acylate solution to be case is preferably from −10 to 55° C., more preferably from 25 to 50° C. In this case, the solution temperature may be the same throughout the entire process, or may differ in different sites of the process. In case where the temperature differs in different sites, the dope shall have the desired temperature just before cast.

### <Stretching Treatment>

The production method of the invention includes a step of stretching the plastic film that contains the film to be the outermost layer thereof, in the direction perpendicular to the film transporting direction by from 20 to 50% at a stretching temperature A±10° C. (wherein A means the temperature at which tan δ of the dynamic viscoelasticity of the thermoplastic resin having a residual solvent content of 0% shows a peak). As described above, the film of the invention has a high total haze and has a low internal haze, and the film of the type can be attained by stretching a plastic film that has, as at least one outermost layer thereof, the film formed by casting a dope containing a thermoplastic resin and inorganic fine particles in an amount of at least 0.1% by mass relative to the thermoplastic resin, under the above-mentioned specific condition to thereby impart the necessary property to the resulting film.

In the film production method of the invention, the film stretching direction under the above-mentioned specific condition is the direction perpendicular to the film transporting direction (transverse direction TD); however, not contradictory to the scope and the sprit of the invention, the film may be stretched in the film transporting direction.

The above-mentioned “A” means the temperature at which tan δ of the dynamic viscoelasticity of the cellulose acylate having a residual solvent content of 0%, as measured with Vibron, shows a peak, and the temperature is intrinsic to each film. Vibron to be used in measuring the dynamic viscoelasticity is not specifically defined. For example, IT Instrument and Control's trade name, DVA200 can be used here.

In the production method of the invention, preferably, a cellulose acylate resin is used as the thermoplastic resin and the film is stretched by from 20 to 40% in the direction perpendicular to the film transporting direction in the stretching step, more preferably, by from 30 to 40% in the direction perpendicular to the film transporting direction.

The stretching method in the direction perpendicular to the film transporting direction is described, for example, in JP-A 62-115035, 4-152125, 4-284211, 4-298310, 11-48271, etc. In the case where the film is stretched in the film transporting direction (in the machine direction), for example, the speed of the film transporting rollers is so regulated that the film winding speed is higher than the film releasing speed, whereby the film is stretched in the intended manner. In the case where the film is stretched in the direction perpendicular to the film transporting direction, the film is conveyed while held by a tenter, and the tenter width is gradually broadened whereby the film may be stretched. After dried, the film may be stretched with a stretcher (preferably, in a mode of monoaxial stretching with a Long stretcher).

In case where the plastic film is used as a protective film for a polarizing element, the transmission axis of the polarizing element must be in parallel to the in-plane slow axis of the plastic film so as to prevent the light leakage in oblique directions to the polarizer. The transmission axis of the roll film-type polarizing element that is produced continuously is generally parallel to the cross direction of the roll film, and therefore, in continuously sticking the roll film-type polarizing element and a protective film of a roll film-type plastic film, the in-plane slow axis of the roll film-type protective film must be parallel to the cross direction of the film. Accordingly, the film is preferably stretched to a larger extend in the cross direction. The stretching treatment may be attained during the course of the film formation process, or the wound film may be unwound and stretched. In the production method of the invention, the film is stretched while it contains the residual solvent therein, and therefore the film is preferably stretched during the course of the film formation process.

### <Drying>

For drying the dope on a metal support in production of a plastic film, generally employable is a method of applying hot air to the surface of the metal support (drum or belt), or that is, on the surface of the web an the metal support; a method of applying hot air to the back of the drum or belt; or a back side liquid heat transfer method that comprises contacting a temperature-controlled liquid with the opposite side of the dope-cast surface of the belt or drum, or that is, the back of the belt or drum to thereby heat the belt or drum by heat transmission to control the surface temperature thereof. Preferred is the backside liquid heat transfer method. The surface temperature of the metal support before the dope is cast thereon may be any degree so far as it is not higher than the boiling point of the solvent used in the dope. However, for promoting the drying or for making the dope lose its flowability on the metal support, preferably, the temperature is set to be lower by from 1 to 10° C. than the boiling point of the solvent having the lowest boiling point of all the solvents in the dope. In case where the cast dope is peeled off after cooled but not dried, then this shall not apply thereto.

For controlling the thickness of the film, the solid concentration in the dope, the slit gap of the die nozzle, the extrusion pressure from the die, and the metal support speed may be suitably regulated so that the formed film could have a desired thickness.

### <Rolling Up>

The plastic film produced in the manner as above is preferably rolled up so that the length of the plastic film is preferably from 100 to 10000 m per roll, more preferably from 50 to 7000 m, even more preferably from 1000 to 6000m. The width of the plastic film is preferably from 0.5 to 5.0 m, more preferably from 1.0 to 3.0 m, even more preferably from 1.0 to 2.5m. In winding the film, preferably, at least one edge thereof is knurled, and the knurling width is preferably from 3 mm to 50 mm, more preferably from 5 mm to 30 mm, and the knurling height is preferably from 0.5 to 500 μm, more preferably from 1 to 200 μm. This may be one-way or double-way knurling.

In general, in large-panel display devices, contrast reduction and color shift may be remarkable in oblique directions; and therefore the film of the invention is especially suitable for use in large-panel display devices. In case where the film of the invention is used as an optical compensatory film for large-panel liquid crystal display devices, for example, the film is shaped to have a width of at least 1470 mm. The optical compensatory film of the invention includes not only film sheets cut to have a size that may be directly incorporated in liquid crystal display devices but also long films continuously produced and rolled up into rolls. The optical compensatory film of the latter embodiment is stored and transported in the rolled form, and is cut into a desired size when it is actually incorporated into a liquid crystal display device or when it is stuck to a polarizing element or the like. The long film may be stuck to a polarizing element formed of a long polyvinyl alcohol film directly as they are, and then when this is actually incorporated into a liquid crystal display device, it may be cut into a desired size. One embodiment of the long optical compensatory film rolled up into a roll may have a length of 2500 m/roll or more.

### EXAMPLES

The invention is described more concretely with reference to the following Examples. In the following Examples, the materials, the reagents and the substances used, their amount and ratio, the details of the treatment and the treatment process may be suitably modified or changed not overstepping the sprit and the scope of the invention. Accordingly, the invention should not be limitatively interpreted by the Examples mentioned below.

In the invention, the film samples were analyzed according to the following measuring methods.

### (Total Haze)

For measuring the total haze thereof, a film sample, 40 mm×80 mm of the invention was analyzed with a haze meter (Suga Test Instruments' “HGM-2DP”) at 25° C. and a relative humidity of 60%, according to JIS K-6714. The results are shown in Table 3 below.

### (Internal Haze)

First, a film sample, 40 mm×80 mm of the invention was analyzed with an Abbe's refractometer (Atago's “Abbe Refractometer 2-T”) to measure its refractive index.

Next, a few drops of an oil having a refractive index falling within a range of “refractive index±0.02” of the thermoplastic resin of which the content was the largest in the film, were applied to the film sample, then the sample was sandwiched between two glass plates (Microslide Glass Model Number S 9111, by Matsunami) each having a thickness of 1 mm, and in that manner the two glass plates and the film were completely optically adhered to each other. In the condition where the total haze was removed, the sample was analyzed with the haze meter (Suga Test Instruments' “HGM-2DP”) according to JIB K-6714. Apart from this, only the oil was sandwiched between the two glass plates and the haze thereof was measured. The haze of the oil alone was subtracted from the haze of the film, and this is the internal haze of the film sample.

The film samples comprising a cellulose aoylate resin of Examples and Comparative Examples had a refractive index of from 1.48 to 1.49, and therefore, they were analyzed using a liquid paraffin having a refractive index of 1.48. The film samples comprising ARTON had a refractive index of 1.52, and were analyzed using a silicone oil having a refractive index of 1.52.

In the invention, one sample was analyzed for a total of 30 times in one and the same measuring method as above, and the data were averaged to give the internal haze of the sample. The results are shown in Table 3 below.

### (Surface Roughness)

Using Anritsu's film thickness gauge, KG601G, a 5-meter film was analyzed at a pitch of 0.1 mm, and the standard deviation of the measured data was computed. The value is represented by σ, and 6σ is the film surface roughness. The results are shown in Table 3 below.

### (Optical Expressibility)

Re and Rth of the film were measured at a wavelength of 590 nm according to the above-mentioned method, using KOBRA 21ADH (by Oji Scientific Instruments). The results are shown in Table 3 below.

### (Film Handling Characteristics During Transport)

The film was checked for the film handling characteristics during transport according to the method mentioned below.

A film sample A, 100 mm×200 mm, and a film sample B, 75 mm×100 mm were conditioned at 23° C. and a relative humidity of 65% for 2 hours. Using a Tensilon tensile tester (RTA-100, by Orientec), the film sample A was fixed on a sample bed, and the film sample B weighted with a 200 g weight (W) was put on the film sample A. The film sample B was pulled in the horizontal direction, and the force (F) by which it began to move was measured. According to the following formula, the static friction coefficient (μ) was computed.

F=μ×W (W is the weight (kgf).

Analyzed as above, the samples were classified into the following groups. The results were evaluated according to the standards mentioned below; and the results are shown in Table 3.

A: 0.7 or less.
B: from more than 0.7 to 0.8.
C: from more than 0.8 to 1.0.
D: more than 1.0.

### [A: Co-Cast Film]

In Examples and Comparative Examples demonstrating the formation of a film composed of at least two layers, the plastic film was produced according to the method mentioned below.

### (1) Preparation of Cellulose Acylate Resin by Synthesis:

A cellulose acylate having a degree of acyl substitution shown in Table 3 was prepared. As a catalyst, sulfuric acid (in an amount of 7.8 parts by mass relative to 100 parts by mass of cellulose) was added, and a carboxylic acid was added for acylation at 40° C. Subsequently, the sulfuric acid catalyst amount, the water amount and the ripening time were controlled to thereby control the degree of total substitution and the degree of 6-position substitution. The ripening temperature was 40° C. Further, the low-molecular weight ingredient of the cellulose acylate was removed by washing with acetone.

### (2) Preparation of Dope:

### <2-1> Core Layer Dope:

The ingredients mentioned were put into a mixing tank, stirred and dissolved, then heated at 90° C. for about 10 minutes. Subsequently, the mixture was filtered through a paper filter having a mean pore size of 34 μm and through a sintered metal filter having a mean pore size of 10 μm.

### Core Layer Dope of Example 1

| | |
|---|---|
| Cellulose acetate | 100.0 parts by mass |
| (having a degree of | |
| substitution of 2.41) | |
| Compound A | 18.5 parts by mass |
| Methylene chloride | 365.5 parts by mass |
| Methanol | 54.6 parts by mass |

Other core layer dopes were produced in the same manner as that for the core layer dope of Example 1, except that the degree of total acyl substitution of the cellulose acylate resin, the type of the additive and the amount of the additive were changed as in Table 3 below. The cellulose acetate propionate resin having a degree of total acyl substitution of 2.39 had a degree of propionyl substitution of 0.8; and the cellulose acetate propionate resin having a degree of total acyl substitution of 2.43 had a degree of propionyl substitution of 0.8.

The additives A to E and H to J are shown in Table 1 below. The additive F is a plasticizer, TPP/BDP; and the additive AO is an antioxidant, Ir1010 (trade name, Irganox 1010 by BASF). In Table 1 below, TPA means terephthalic acid; PA means phthalic acid; AA means adipic acid; and SA means succinic acid.

The amount of the additive is in terms of % by mass relative to the thermoplastic resin in each core layer dope.

TABLE 1

| | Glycol Unit | | | | | | | | | | | | |
|---|---|---|---|---|---|---|---|---|---|---|---|---|---|
| | hydroxyl | | | | | | | | | | | | |
| | group | | | | | | | | | | | | |
| | blocking | | | 1,4- | 1,5- | 2,10- | | Dicarboxylic Acid Unit | | | | | Viscosity- |
| | ratio at | ethylene | propylene | butane- | pentane- | decane- | Mean | | | | | Mean | Average |
| | both ends | glycol | glycol | diol | diol | diol | Carbon | TPA | PA | AA | SA | Carbon | Molecular |
| | (%) | (%) | (%) | (%) | (%) | (%) | Number | (mol %) | (mol %) | (mol %) | (mol %) | Number | Weight |
| Additive A | 100 | 50 | 50 | 0 | 0 | 0 | 2.5 | 55 | 0 | 0 | 45 | 6.2 | 730 |
| Additive B | 100 | 100 | 0 | 0 | 0 | 0 | 2.0 | 45 | 5 | 20 | 30 | 6.0 | 840 |
| Additive C | 0 | 25 | 75 | 0 | 0 | 0 | 2.75 | 45 | 10 | 0 | 45 | 6.2 | 690 |
| Additive D | 0 | 50 | 50 | 0 | 0 | 0 | 2.5 | 55 | 0 | 0 | 45 | 6.2 | 690 |
| Additive E | 0 | 100 | 0 | 0 | 0 | 0 | 2.0 | 45 | 5 | 20 | 30 | 6.0 | 680 |
| Additive H | 100 | 50 | 0 | 50 | 0 | 0 | 3.0 | 50 | 0 | 0 | 50 | 6.0 | 800 |
| Additive I | 100 | 50 | 0 | 0 | 50 | 0 | 3.5 | 50 | 0 | 0 | 50 | 6.0 | 800 |
| Additive J | 100 | 50 | 0 | 0 | 0 | 50 | 6.0 | 50 | 0 | 0 | 50 | 6.0 | 800 |

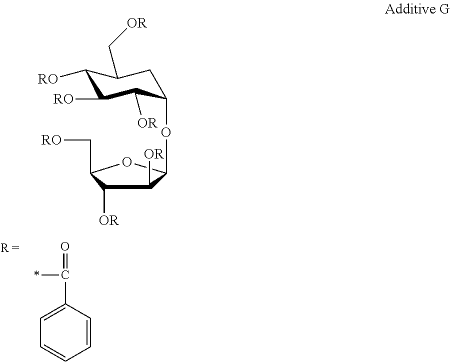

### <2-2> Surface Layer Cellulose Acylate Dope:

The ingredients mentioned were put into a mixing tank, stirred and dissolved, then heated at 90° C. for about 10 minutes. Subsequently, the mixture was filtered through a paper filter having a mean pore size of 34 μm and through a sintered metal filter having a mean pore size of 10 μm.

### Surface Layer Dope of Example 1

| | |
|---|---|
| Cellulose acetate | 100.0 parts by mass |
| (having a degree of | |
| substitution of 2.81) | |
| Compound A | 11.0 parts by mass |
| Inorganic fine particles | 0.2 parts by mass |
| (Aerosil R972) | |
| Methylene chloride | 365.5 parts by mass |
| Methanol | 54.6 parts by mass |

TABLE 2

| | | Specific | Mean Size | Apparent |
|---|---|---|---|---|
| | | Surface Area, | of Primary | Specific |
| | | BET Method | Particles | Gravity |
| Grade | | m²/g | nm | g/l |
| R972 | hydrophobic | 110 ± 20 | about 16 | about 50 |
| R972V | hydrophobic | 110 ± 20 | about 16 | about 90 |
| R974 | hydrophobic | 170 ± 20 | about 12 | about 50 |
| R976 | hydrophobic | 190 ± 20 | about 10 | about 40 |

Other surface layer dopes were produced in the same manner as that for the surface layer dope of Example 1, except that the degree of total acyl substitution of the cellulose acylate resin, the type of the additive and the amount of the additive were changed as in Table 3 below.

### (3) Co-Casting:

The core layer dope and the surface layer dope were simultaneously co-cast so as to form the core layer and the surface layer in the thickness ratio as in Table 3 below. The band used here was formed of SUS.

### (4) Stretching:

The formed web (film) was peeled from the band and clipped, and while the residual solvent amount in the film was from 30 to 5% by mass relative to the whole film, this was stretched under the condition of edge-fixed monoaxial stretching, at the stretching temperature to the stretching draw ratio shown in Table 3, in the direction perpendicular to the film transporting direction (lateral direction), using a tenter.

Subsequently, the film was unclipped, and dried at 130° C. for 20 minutes. In this step, the casting film thickness was so controlled that the stretched film thickness could be the thickness (unit, μm) shown in Table 3. For producing the films having the composition shown in Table 3 and for determining the production aptitude of the films, at least 24 rolls of each film having a width of 1280 mm and a length of 2600 mm were produced under the above-mentioned condition. Of 24 rolls thus continuously produced, the film of one roll was sampled at intervals of 1001n to give film samples each having a length of 1 m (and having a width of 1280 mm). The film samples were tested and analyzed.

### [B: Single Layer Film]

A single layer film was produced. In other Examples and Comparative Examples, plastic films were produced according to the method mentioned below.

### (1) Preparation of Cellulose Acylate Resin:

A cellulose acylate resin was prepared in the same manner as that in production of co-cast films.

### (2) Preparation of Cyclic Olefin Resin:

ARTON (JSR's trade name, model number, D4531) was used as a cyclic olefin resin. This was pre-treated through dissolution in dichloromethane, prior to preparing the dope.

### (3) Preparation of Dope:

### <3-1> Thermoplastic Resin Solution:

The ingredients mentioned were put into a mixing tank, stirred and dissolved, then heated at 90° C. for about 10 minutes. Subsequently, the mixture was filtered through a paper filter having a mean pore size of 34 μm and through a sintered metal filter having a mean pore size of 10 μm.

### Cellulose Acylate Solution in Comparative Example 1

| | |
|---|---|
| Thermoplastic resin | 100.0 parts by mass |
| shown in Table 3 below | |
| Additive A shown | 18.5 parts by mass |
| in Table 3 below | |
| Methylene chloride | 403.0 parts by mass |
| Methanol | 60.2 parts by mass |

### <3-2> Matting Agent Dispersion:

The following composition containing the cellulose acylate solution prepared in the above was put into a disperser to prepare a matting agent dispersion.

### Matting Agent Dispersion in Comparative Example 1

| | |
|---|---|
| Inorganic fine particles | 0.2 parts by mass |
| (Aerosil R972) | |
| Methylene chloride | 72.4 parts by mass |
| Methanol | 10.8 parts by mass |
| Cellulose acylate solution | 10.3 parts by mass |

100 parts by mass of the cellulose acylate solution in Comparative Example 1 and the matting dispersion in Comparative Example 1 in an amount of 0.03 parts by mass of the inorganic fine particles therein relative to the cellulose acylate resin were mixed to prepare a dope for film formation.

Other dopes were produced in the same manner as that for the dope of Comparative Example 1, except that the type of the thermoplastic resin, the degree of total acyl substitution of the cellulose acylate resin in the case where the thermoplastic resin is a cellulose acylate resin, the type of the additive and the amount of the additive were changed as in Table 3 below.

### (4) Casting:

Using a band caster, the above-mentioned dope was cast to form a film having a thickness as in Table 3 below. The band used here was formed of SUS.

### (5) Stretching:

The formed web (film) was peeled from the band and clipped, and while the residual solvent amount in the film was from 30 to 5% by mass relative to the whole film, this was stretched under the condition of edge-fixed monoaxial stretching, at the stretching temperature to the stretching draw ratio shown in Table 3, in the direction perpendicular to the film transporting direction (lateral direction), using a tenter.

Subsequently, the film was unclipped, and dried at 130° C. for 20 minutes. In this step, the casting film thickness was so controlled that the stretched film thickness could be the thickness (unit, μm) shown in Table 3. For producing the films having the composition shown in Table 3 and for determining the production aptitude of the films, at least 24 rolls of each film having a width of 1280 nun and a length of 2600 mm were produced under the above-mentioned condition. Of 24 rolls thus continuously produced, the film of one roll was sampled at intervals of 100 m to give film samples each having a length of 1 m (and having a width of 1280 mm). The film samples were tested and analyzed.

### [C: Production of Polarizer]

Iodine was adsorbed by a stretched polyvinyl alcohol film to prepare a polarizing element. The film of Examples and Comparative Examples produced herein was stuck to one side of the polarizing element, using a polyvinyl alcohol adhesive. The saponification was attained under the condition mentioned below.

An aqueous solution of sodium hydroxide (1.5 mol/L) was prepared and kept at 55° C. An aqueous solution of diluted sulfuric acid (0.005 mol/L) was prepared and kept at 35° C. The film produced in Examples and Comparative Examples was dipped in the aqueous sodium hydroxide solution for 2 minutes, and then dipped in water to fully remove the aqueous sodium hydroxide solution. Next, this was dipped in the aqueous diluted sulfuric acid solution for 1 minute, and dipped in water to fully remove the aqueous diluted sulfuric acid solution. Finally, the sample was fully dried at 120° C.

A commercially-available cellulose triacetate film (Fujitac TD80UF, by FUJIFILM) was saponified, and stuck to the opposite side of the polarizing element, using a polyvinyl alcohol adhesive, and dried at 70° C. for at least 10 minutes.

The polarizing element and the film of Examples and Comparative Examples were so arranged that the transmission axis of the former could be parallel to the slow axis of the latter. The polarizing element and the commercial film were so arranged that the transmission axis of the former could be perpendicular to the slow axis of the latter.

(Production of Polarizer and Contrast of Display Device with the Polarizer)

The polarizer was peeled away from the liquid crystal cell of BRAVIA-KDL40V5 (trade name by Sony). An adhesive was applied to the polarizer produced in Examples and Comparative Examples, on side of the film of Examples and Comparative Examples and opposite to the polarizing element stuck thereto, and the polarizer was stuck to the liquid crystal cell in the order of film of Example or Comparative Example/adhesive/liquid crystal cell. The contrast of the liquid crystal display device was measured. The results are shown in Table 3 below.

TABLE 3

| | Stretching Condition | | | | | | | | | | | | | |
|---|---|---|---|---|---|---|---|---|---|---|---|---|---|---|
| | | | | Concentration of | | | | | | | | | | |
| | | | | Inorganic Fine | | | | | | | | | | |
| | | | | Particles Relative | Amount of | | Film Properties | | | | | | | |
| | | | | to the | Inorganic Fine | | | | | | | | Film | |
| | | Film tanδ | | Thermoplastic | Particles relative to | | | | | | | | Handling | CR of |
| | Stretching | Peak | | Resin in the | the thermoplastic | Total Film | | | Film | Surface | | | Characteristics | Display |
| | Temperature | Temperature A | Draw Ratio in | Outermost Layer | resin in the film | Thickness | | Internal | Refractive | Roughness | Re | Rth | during | Device |
| | (° C.) | (° C.) | TD Stretching | (% by mass) | (% by mass) | (mm) | Total Haze | Haze | Index | (μm) | (nm) | (nm) | Transport | with Film |
| Comparative Example 1 | 176 | 176 | 1.29 | 0.03 | 0.03 | 85 | 0.2 | 0 | 1.49 | 0.4 | 49 | 119.5 | C | 7000 |
| Example 1 | 175 | 176 | 1.29 | 0.2 | 0.003 | 59 | 0.41 | 0 | 1.49 | 0.71 | 48 | 119 | B | 7000 |
| Example 2 | 172 | 176 | 1.32 | 0.11 | 0.005 | 59 | 0.52 | 0.02 | 1.49 | 0.75 | 48 | 119 | A | 7000 |
| Comparative Example 2 | 188 | 176 | 1.32 | 0.11 | 0.005 | 59 | 0.32 | 0.02 | 1.49 | 0.45 | 49 | 85 | C | 5500 |
| Example 3 | 172 | 178 | 1.32 | 0.11 | 0.005 | 59 | 0.55 | 0.02 | 1.48 | 0.77 | 48 | 119 | A | 7000 |
| Example 4 | 172 | 176 | 1.32 | 0.11 | 0.005 | 59 | 0.55 | 0.02 | 1.48 | 0.78 | 48 | 119 | A | 7000 |
| Example 5 | 172 | 176 | 1.32 | 0.11 | 0.005 | 59 | 0.51 | 0.02 | 1.48 | 0.7 | 48 | 119 | A | 7000 |
| Example 6 | 175 | 176 | 1.34 | 0.2 | 0.004 | 55 | 0.51 | 0 | 1.48 | 0.78 | 55 | 120 | A | 7000 |
| Example 7 | 172 | 176 | 1.32 | 0.2 | 0.2 | 59 | 0.51 | 0.04 | 1.49 | 0.77 | 52 | 119 | A | 8440 |
| Example 8 | 167 | 176 | 1.25 | 0.12 | 0.002 | 72 | 0.51 | 0.04 | 1.49 | 0.71 | 55 | 220 | A | 7000 |
| Example 9 | 165 | 172 | 1.35 | 0.15 | 0.005 | 60 | 0.42 | 0.04 | 1.49 | 0.55 | 60 | 160 | B | 8800 |
| Example 10 | 175 | 176 | 1.35 | 0.2 | 0.007 | 60 | 0.55 | 0.04 | 1.49 | 0.7 | 50 | 110 | A | 8800 |
| Example 11 | 182 | 182 | 1.35 | 0.15 | 0.005 | 60 | 0.42 | 0.04 | 1.49 | 0.75 | 40 | 95 | B | 8800 |
| Example 12 | 172 | 175 | 1.32 | 0.12 | 0.002 | 59 | 0.53 | 0.03 | 1.49 | 0.71 | 52 | 119 | A | 7000 |
| Example 13 | 172 | 176 | 1.38 | 0.12 | 0.002 | 59 | 0.51 | 0.04 | 1.49 | 0.71 | 52 | 118 | A | 7000 |
| Example 14 | 172 | 177 | 1.32 | 0.12 | 0.002 | 59 | 0.53 | 0.05 | 1.49 | 0.71 | 52 | 119 | A | 7000 |
| Example 15 | 172 | 176 | 1.38 | 0.12 | 0.002 | 59 | 0.51 | 0.04 | 1.49 | 0.71 | 52 | 120 | A | 7000 |
| Example 16 | 170 | 176 | 1.32 | 0.2 | 0.2 | 59 | 0.62 | 0.05 | 1.49 | 0.84 | 53 | 126 | A | 6300 |
| Example 17 | 168 | 176 | 1.32 | 0.2 | 0.2 | 59 | 0.55 | 0.07 | 1.49 | 0.91 | 54 | 130 | A | 6020 |
| Comparative Example 3 | 165 | 176 | 1.32 | 0.2 | 0.2 | 59 | 0.54 | 0.09 | 1.49 | 0.91 | 55 | 135 | A | 5740 |
| Example 18 | 170 | 165 | 1.33 | 0.15 | 0.15 | 58 | 0.45 | 0.04 | 1.48 | 0.75 | 51 | 118 | B | 6500 |
| Example 19 | 170 | 165 | 1.33 | 0.15 | 0.15 | 58 | 0.54 | 0.05 | 1.48 | 0.75 | 51 | 118 | A | 6300 |
| Example 20 | 165 | 170 | 1.33 | 0.18 | 0.18 | 41 | 0.51 | 0.04 | 1.48 | 0.75 | 49 | 119 | A | 6300 |
| Example 21 | 165 | 170 | 1.33 | 0.18 | 0.002 | 41 | 0.45 | 0.04 | 1.48 | 0.75 | 49 | 119 | B | 6300 |
| Comparative Example 4 | 158 | 151 | 1.52 | 0 | 0 | 40 | 0.05 | 0.01 | 1.52 | 0.23 | 50 | 110 | C | 6860 |
| Example 22 | 158 | 151 | 1.52 | 0.3 | 0.3 | 40 | 0.8 | 0.03 | 1.52 | 0.75 | 50 | 110 | A | 6580 |
| Example 23 | 170 | 165 | 1.35 | 0.11 | 0.004 | 58 | 0.51 | 0.02 | 1.48 | 0.75 | 51 | 120 | A | 6800 |
| Example 24 | 165 | 160 | 1.35 | 0.11 | 0.004 | 58 | 0.55 | 0.03 | 1.49 | 0.78 | 48 | 122 | A | 6800 |
| Example 25 | 163 | 168 | 1.35 | 0.11 | 0.004 | 58 | 0.7 | 0.04 | 1.49 | 0.85 | 45 | 115 | A | 6800 |
| Comparative Example 5 | 130 | 151 | 1.2 | 0.15 | 0.011 | 80 | 0.7 | 0.12 | 1.48 | 0.75 | 96 | 112 | A | 5320 |

| | Core Layer Dope | | | | | | Surface Layer Dope | | | | | | |
|---|---|---|---|---|---|---|---|---|---|---|---|---|---|
| | | | Inorganic Fine Particles | | | | | | Inorganic Fine Particles | | | | |
| | | | | Amount Added | | | | | | Amount Added | | | |
| | | | | Relative to the | | | | | | Relative to the | | | |
| | Thermoplastic Resin 1 | | | Thermoplastic | Other Additive | | Thermoplastic Resin 2 | | | Thermoplastic | Other Additive | | Total |
| | | Degree | | Resin in the | | Amount | | Degree | | Resin in the | | Amount | Thickness of |
| | | of | | Core Layer | | Added | | of | | Surface Layer | | Added | Both Surface |
| | | Total Acyl | | Dope | | (part by | | Total Acyl | | Dope | | (parts by | Layers |
| | Type | Substitution | Type | (% by mass) | Type | mass) | Type | Substitution | Type | (% by mass) | Type | mass) | (mm) |
| Comparative Example 1 | acetate | 2.41 | R972 | 0.03 | A | 18.5 | — | — | — | 0 | — | 0 | 0 |
| Example 1 | acetate | 2.41 | — | 0 | A | 18.5 | acetate | 2.81 | R972 | 0.2 | A | 11 | 1 |
| Example 2 | acetate | 2.42 | — | 0 | A | 18.5 | acetate | 2.81 | R972 | 0.11 | A | 11 | 2.5 |
| Comparative Example 2 | acetate | 2.42 | — | 0 | A | 18.5 | acetate | 2.81 | R972 | 0.11 | A | 11 | 2.5 |
| Example 3 | acetate | 2.42 | — | 0 | A | 18.5 | acetate | 2.81 | R972V | 0.11 | A | 11 | 2.5 |
| Example 4 | acetate | 2.42 | — | 0 | A | 18.5 | acetate | 2.81 | R974 | 0.11 | A | 11 | 2.5 |
| Example 5 | acetate | 2.42 | — | 0 | A | 18.5 | acetate | 2.81 | R976 | 0.11 | A | 11 | 2.5 |
| Example 6 | acetate | 2.41 | — | 0 | A | 18.5 | acetate | 2.81 | R972 | 0.2 | A | 11 | 1 |
| Example 7 | acetate | 2.41 | R972 | 0.2 | A | 18.5 | — | — | — | 0 | — | 0 | 0 |
| Example 8 | acetate | 2.41 | — | 0 | A | 12 | acetate | 2.81 | R972 | 0.12 | A | 11 | 1 |
| Example 9 | acetate | 2.05 | — | 0 | A | 18.5 | acetate | 2.05 | R972 | 0.15 | A | 11 | 2 |
| Example 10 | acetate | 2.5 | — | 0 | A | 18.5 | acetate | 2.5 | R972 | 0.2 | A | 11 | 2 |
| Example 11 | acetate | 2.65 | — | 0 | A | 18.5 | acetate | 2.65 | R972 | 0.15 | A | 11 | 2 |
| Example 12 | acetate | 2.42 | — | 0 | B | 18.5 | acetate | 2.81 | R972 | 0.12 | B | 11 | 1 |
| Example 13 | acetate | 2.43 | — | 0 | C | 18.5 | acetate | 2.81 | R972 | 0.12 | C | 11 | 1 |
| Example 14 | acetate | 2.41 | — | 0 | D | 18.5 | acetate | 2.81 | R972 | 0.12 | D | 11 | 1 |
| Example 15 | acetate | 2.42 | — | 0 | E + F | 19 + 2 | acetate | 2.81 | R972 | 0.12 | E + F | 10 + 1 | 1 |
| Example 16 | acetate | 2.41 | R972 | 0.2 | A | 18.5 | — | — | — | 0 | — | 0 | 0 |
| Example 17 | acetate | 2.41 | R972 | 0.2 | A | 18.5 | — | — | — | 0 | — | 0 | 0 |
| Comparative Example 3 | acetate | 2.41 | R972 | 0.2 | A | 18.5 | — | — | — | 0 | — | 0 | 0 |
| Example 18 | propionate | 2.39 | R972 | 0.15 | A | 18.5 | — | — | — | 0 | — | 0 | 0 |
| Example 19 | propionate | 2.39 | R972 | 0.15 | A | 18.5 | propionate | 2.65 | R972 | 0.18 | A | 11 | 0.5 |
| Example 20 | propionate | 2.43 | R972 | 0.18 | G | 8 | — | — | — | 0 | — | 0 | 0 |
| Example 21 | propionate | 2.43 | — | 0 | G | 8 | propionate | 2.82 | R972 | 0.18 | G | 11 | 0.5 |
| Comparative Example 4 | ARTON | — | R972 | 0 | AO | 0.3 | — | — | — | 0 | — | 0 | 0 |
| Example 22 | ARTON | — | R972 | 0.3 | AO | 0.3 | — | — | — | 0 | — | 0 | 0 |
| Example 23 | acetate | 2.42 | R972 | 0 | H | 19 | acetate | 2.81 | R972 | 0.12 | H | 11 | 2 |
| Example 24 | acetate | 2.42 | R972 | 0 | I | 19 | acetate | 2.81 | R972 | 0.12 | I | 11 | 2 |
| Example 25 | acetate | 2.42 | R972 | 0 | J | 19 | acetate | 2.81 | R972 | 0.12 | J | 11 | 2 |
| Comparative Example 5 | acetate | 2.86 | — | 0 | B + C | 5.4 + 2.2 | acetate | 2.86 | R972 | 0.15 | B + C | 5 + 4 | 6 |

From Table 3, it is known that the films of the invention have good film handling characteristics during transport and that, when the film is worked into a polarizer and incorporated in a display device, the display device having the film of the invention has a high contrast. On the other hand, in the film of Comparative Example 1, the concentration of the inorganic fine particles added to the thermoplastic resin in the outermost layer is lower than the range in the production method of the invention, and it is known that the total haze of the film is lower than the range of the invention and the film handling characteristics during transport is poor. The film of Comparative Example 2 was stretched at the stretching temperature higher than the range in the production method of the invention, and it is known that the total haze of the film is low and the film handling characteristics during transport is poor and, in addition, the contrast of the display device comprising the polarizer with the film is low. The film of Comparative Example 3 was stretched at the stretching temperature lower than the range in the production method of the invention, and it is known that the internal haze of the film is higher than the range of the invention and, in addition, the contrast of the display device comprising the polarizer with the film is low. The film of Comparative Example 4 does not contain inorganic fine particles, and it is known that the total haze of the film is lower than the range of the invention and the film handling characteristics during transport is poor. The film of Comparative Example 5 was produced according to the method described in JP-A 2008-262161 in which the stretching temperature was much lower than the range of the invention, and it is known that the internal haze of the film is outside the scope of the invention and the contrast of the display device comprising the polarizer with the film is low.

In addition, it was confirmed that the films of the invention shown in Table 3 all had good adhesiveness, and could be saponified for a shorter period of time.

(Contrast Change with Time)

This is for investigating the influence of the additive on the durability of films. The films of Examples 2, 12 to 15 and 23 to 25 were selected, and statically kept in an environment at 60° C. and a relative humidity of 90% for 500 hours. Afterwards, the contrast was measured with each film, in accordance with the same process as above (for production of polarizer and contrast of display device with the polarizer). The reduction in the contrast of the aged sample as compared with the sample not aged for 500 hours was computed, and the results are shown in Table 4 below.

TABLE 4

| | Additive | | | |
|---|---|---|---|---|
| | | | Mean | Contrast |
| | | | Carbon | Reduction |
| | Additive | Amount | Number | (%) |
| Example 2 | A | 18.5 | 2.5 | 0.7 |
| Example 12 | B | 18.5 | 2.0 | 11.4 |
| Example 13 | C | 18.5 | 2.75 | 0.0 |
| Example 14 | D | 18.5 | 2.5 | 1.4 |
| Example 15 | E + F | 19 + 2 | 2.0 | 12.8 |
| Example 23 | H | 18.5 | 3.0 | 0.0 |
| Example 24 | I | 18.5 | 3.5 | 0.0 |
| Example 25 | J | 18.5 | 6.0 | 0.0 |

The contrast reduction with the film was all within a practicable range. Above all, the contrast reduction of the display device with the film of Examples 2, 13, 14, 23, 24 and 25 to which the additive added had a mean carbon number of at least 2.4 was low; and the contrast of the display device with the film of Examples 13, 23, 24 and 25 to which the additive added had a mean carbon number of at least 2.6 did not lower. The results confirm extremely high durability of the films of the invention.

The present disclosure relates to the subject matter contained in Japanese Patent Application No. 2009-267390, filed on Nov. 25, 2009, and Japanese Patent Application No. 2010-217619, filed on Sep. 28, 2010, the contents of which are expressly incorporated herein by reference in their entirety. All the publications referred to in the present specification are also expressly incorporated herein by reference in their entirety.

The foregoing description of preferred embodiments of the invention has been presented for purposes of illustration and description, and is not intended to be exhaustive or to limit the invention to the precise form disclosed. The description was selected to best explain the principles of the invention and their practical application to enable others skilled in the art to best utilize the invention in various embodiments and various modifications as are suited to the particular use contemplated. It is intended that the scope of the invention not be limited by the specification, but be defined claims set forth below.

What is claimed is:

1. A plastic film having an internal haze of at most 0.08 and a total haze of at least 0.41.

2. The plastic film according to claim 1, wherein the total haze is at least 0.51.

3. The plastic film according to claim 1, having a surface roughness of at least 0.7.

4. The plastic film according to claim 1, comprising an ester polymer.

5. The plastic film according to claim 4, wherein the mean carbon number of the diol residue constituting the ester polymer is from 2.3 to 7.0.

6. The plastic film according to claim 1, comprising inorganic fine particles in the outermost layer thereof.

7. The plastic film according to claim 1, comprising a cellulose acylate resin or a cyclic olefin resin.

8. The plastic film according to claim 1, comprising a cellulose acylate resin.

9. The plastic film according to claim 8, wherein the mean value of the degree of total acyl substitution of the cellulose acylate resin is from 2.1 to 2.6.

10. The plastic film according to claim 1, comprising a core layer and at least one surface layer laminated on each of both surfaces of the core layer.

11. The plastic film according to claim 10, produced by co-casting and comprising inorganic fine particles in the surface layer.

12. The plastic film according to claim 10, wherein the cellulose acylate resin contained in the core layer differs from the cellulose acylate resin contained in the surface layer in the degree of total acyl substitution.

13. The plastic film according to claim 12, wherein the cellulose acylate resin constituting the core layer has a degree of total acyl substitution of from 2.1 to 2.6.

14. The plastic film according to claim 10, wherein the core layer does not contain inorganic fine particles at all.

15. A method for producing a plastic film, comprising:
casting a dope that contains a thermoplastic resin and inorganic fine particles in an amount of at least 0.1% by mass relative to the thermoplastic resin, to thereby form a film to be at least one outermost layer of a plastic film, and
stretching the plastic film that contains the film to be the outermost layer thereof, in the direction perpendicular to the film transporting direction by from 20 to 60% at a stretching temperature A±10° C. wherein A means the temperature at which tan δ of the dynamic viscoelasticity of the thermoplastic resin having a residual solvent content of 0% shows a peak.

16. The method for producing a plastic film according to claim 15, wherein the thermoplastic resin is a cellulose acylate resin and, in the stretching step, the film is stretched in the direction perpendicular to the film transporting direction by from 20 to 40%.

17. The method for producing a plastic film according to claim 15, wherein the dope comprising a thermoplastic resin and inorganic fine particles in an amount of at least 0.1% by mass relative to the thermoplastic resin, and at least one core layer dope comprising a thermoplastic resin are cast successively or co-cast simultaneously in such a manner that the dope comprising a thermoplastic resin and inorganic fine particles in an amount of at least 0.1% by mass relative to the thermoplastic resin can be laminated on both surfaces of the core layer dope.

18. The method for producing a plastic film according to claim 15, wherein the dope comprising a thermoplastic resin and inorganic fine particles in an amount of at least 0.1% by mass relative to the thermoplastic resin, and at least one core layer dope comprising a thermoplastic resin are co-cast simultaneously in such a manner that the dope comprising a thermoplastic resin and inorganic fine particles in an amount of at least 0.1% by mass relative to the thermoplastic resin can be laminated on both surfaces of the core layer dope.

19. The method for producing a plastic film according to claim 17, wherein the core layer dope does not contain inorganic fine particles at all.

20. A plastic film produced by:
casting a dope that contains a thermoplastic resin and inorganic fine particles in an amount of at least 0.1% by mass relative to the thermoplastic resin, to thereby form a film to be at least one outermost layer of a plastic film, and
stretching the plastic film that contains the film to be the outermost layer thereof, in the direction perpendicular to the film transporting direction by from 20 to 60% at a stretching temperature A±10° C. wherein A means the temperature at which tan δ of the dynamic viscoelasticity of the thermoplastic resin having a residual solvent content of 0% shows a peak.

21. A polarizer comprising at least one plastic film having an internal haze of at most 0.08 and a total haze of at least 0.41.

22. A liquid crystal display device comprising at least one plastic film having an internal haze of at most 0.08 and a total haze of at least 0.41.
